(19) United States
(12) Patent Application Publication
(10) Pub. No.: US 2017/0261871 A1
(43) Pub. Date: Sep. 14, 2017

(54) ELECTROPHOTOGRAPHIC PHOTORECEPTOR

(71) Applicant: Mitsubishi Chemical Corporation, Chiyoda-ku (JP)

(72) Inventor: Kazutaka IDA, Kanagawa (JP)

(73) Assignee: Mitsubishi Chemical Corporation, Chiyoda-ku (JP)

(21) Appl. No.: 15/606,765

(22) Filed: May 26, 2017

Related U.S. Application Data
(63) Continuation of application No. PCT/JP2015/083248, filed on Nov. 26, 2015.

(30) Foreign Application Priority Data
Nov. 28, 2014 (JP) 2014-241987

(51) Int. Cl. G03G 5/047; C07C 15/14; G03G 5/06; G03G 5/082; G03G 5/147; G03G 5/043

(52) U.S. Cl. CPC G03G 5/047; G03G 5/14713; G03G 5/0436; G03G 5/0607; G03G 5/08214; C07C 15/14

(57) ABSTRACT

The present invention relates to an electrophotographic photoreceptor comprising, on a conductive base: a charge generation layer; and a charge transport layer having a film thickness of 15 μm to 40 μm, wherein the charge transport layer is an outermost layer, and the charge transport layer contains an inorganic filler and a hydrocarbon compound represented by the following Formula (1).

### TECHNICAL FIELD

The present invention relates to an electrophotographic photoreceptor, specifically, and to an electrophotographic photoreceptor which has improved strong-exposure resistance characteristics, is excellent in mechanical characteristics such as abrasion resistance and filming resistance, and is also excellent in gas resistance.

### BACKGROUND ART

Recently, an electrophotographic photoreceptor requires strong durability than before, in both of an aspect of electrical characteristics and an aspect of mechanical characteristics. Among the aspects, in the aspect of mechanical characteristics, improving abrasion resistance on a surface of an outermost layer of a photoreceptor is one object for corresponding to a long-term use. Another object is that a negative influence by so-called strong exposure to the photoreceptor which is performed by natural light, stray light of a fluorescent lamp, or the like in maintenance, is suppressed in order to easily perform required machine maintenance in a using period.

Among the aspect, in the aspect of the former mechanical characteristics, a technology (PTL 1) in which metal oxide particles are caused to be contained in a protective layer, and thus abrasion resistance and the like are improved is known. In the technology, metal oxide particles are caused to be contained in a protective layer which is thinner than a photosensitive layer (by about 1 to 5 μm), and thus it is possible to reduce the used amount of the metal oxide particles. In addition, the technology has an intention of improving mechanical characteristics while a negative influence on electrical characteristics is suppressed. Thus, the technology is a reasonable method.

Resistance against the latter damage to a photoreceptor by strong exposure which is performed by natural light, stray light of a fluorescent lamp, or the like is referred to as strong-exposure resistance characteristics, and as a technology of improving the strong-exposure resistance characteristics, a technology (PTL 2) of improving the strong-exposure resistance characteristics by using a specific compound in a photosensitive layer is known. The specific compound has an electron-donating and modified amino structure.

The essence of this technology is that a specific compound having an electron-donating skeleton is caused to be contained in the photosensitive layer, and thus an action as a filter is conducted, and an occurrence of damage to a photoreceptor, which is applied by the natural light or the stray light of a fluorescent lamp is suppressed. The electron-donating skeleton performs absorption in a wavelength region corresponding to the above light.

An estimated mechanism of the occurrence of damage is as follows. The cause of an occurrence of photoreceptor damage is that the photosensitive layer absorbs the natural light or the stray light of a fluorescent lamp, and thus an excited state of a charge transport material in the photosensitive layer or an excited state of a charge transfer complex between the charge transport material and a binder resin is caused, and a reaction with oxygen or ozone, which continuously occurs, causes degradation of the charge transport material.

The method in which the specific compound is added, and thus absorbing harmful light such as natural light or stray light of a fluorescent lamp by a photosensitive material in the photosensitive layer is suppressed is a reasonable method as the countermeasure for suppressing the occurrence of photoreceptor damage.

### CITATION LIST

### Patent Literature

[PTL 1] JP-A-2002-182416

[PTL 2] JP-A-2004-206109

### SUMMARY OF INVENTION

### Technical Problem

However, in the technology disclosed in PTL 2, in which the specific compound is added, since the specific compound having an electron-donating skeleton is in a compatibilized state in a charge transport layer, trapping in charge transport occurs easily, and, as a result, a change of a potential after strong exposure to a photoreceptor is not sufficiently suppressed.

The present invention is made to solve the above-described problem. That is, an object of the present invention is to provide an electrophotographic photoreceptor in which electrical characteristics by strong exposure fluctuate small, and mechanical characteristics and gas resistance are excellent, and to provide an image forming apparatus having the electrophotographic photoreceptor mounted therein.

### Solution to Problem

The inventors performed close examination for solving the problem by using a material which is not compatible with a photosensitive layer, and thus completed a technology of providing a photoreceptor which has good strong-exposure resistance characteristics, and is also excellent in mechanical characteristics or gas resistance.

That is, the main points of the present invention are included in the following <1> to <12>.

<1> An electrophotographic photoreceptor comprising, on a conductive base: a charge generation layer; and a charge transport layer having a film thickness of 15 μm to 40 μm, wherein the charge transport layer is an outermost layer, and the charge transport layer contains an inorganic filler and a hydrocarbon compound represented by the following Formula (1):

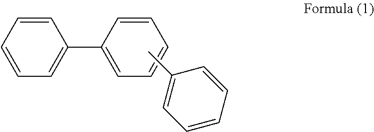

<2> The electrophotographic photoreceptor according to the <1>, wherein the inorganic filler is silica.
<3> The electrophotographic photoreceptor according to the <2>, wherein the silica is subjected to a surface modification.
<4> The electrophotographic photoreceptor according to any one of the <1> to <3>, wherein the inorganic filler has an average primary particle diameter of 0.01 μm to 1 μm.
<5> The electrophotographic photoreceptor according to any one of the <1> to <4>, wherein the charge transport layer contains a binder resin, and a content of the inorganic filler is 5 mass % to 30 mass % with respect to the binder resin.
<6> The electrophotographic photoreceptor according to any one of the <1> to <5>, wherein a percentage of the hydrocarbon compound represented by Formula (1) is 10 mass % to 100 mass %, with respect to the inorganic filler.
<7> The electrophotographic photoreceptor according to any one of the <1> to <6>, wherein the charge transport layer contains an electron attracting compound represented by Formula (2):

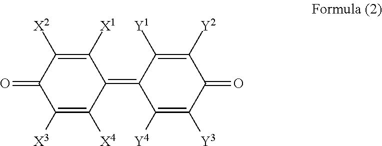

[in Formula (2), X¹, X², X³, X⁴, Y¹, Y², Y³, and Y⁴ each respectively indicate a hydrogen atom, an alkyl group, an aryl group, an acyl group, or a bivalent organic group, and a ring structure including X¹ and X², a ring structure including X³ and X⁴, a ring structure including Y¹ and Y², and a ring structure including Y³ and Y⁴ may be formed]

<8> The electrophotographic photoreceptor according to the <7>, wherein the electron attracting compound represented by Formula (2) is any one of compounds represented by the following Formulas (2a) to (2d):

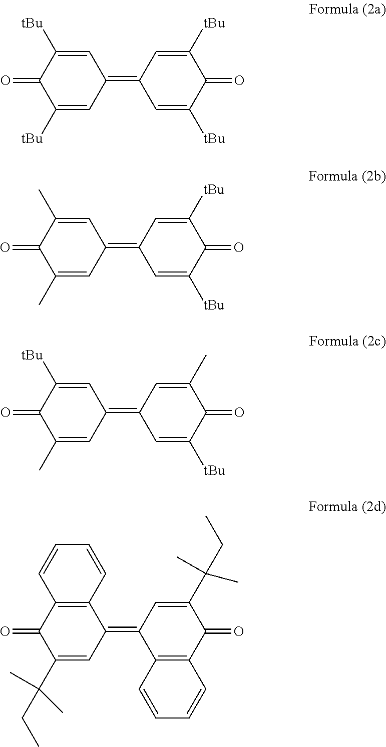

<9> The electrophotographic photoreceptor according to the <7> or <8>, wherein a content percentage of the compound represented by Formula (2) is 2 mass % to 50 mass % with respect to the silica.
<10> The electrophotographic photoreceptor according to any one of the <1> to <9>, wherein the charge generation layer contains D type (Y type) titanyl phthalocyanine in which a clear peak is shown at a Bragg angle 2θ (±0.2°) which is 27.1° to 27.3°, in a CuKα characteristic X-ray diffraction spectrum.
<11> The electrophotographic photoreceptor according to any one of the <1> to <10>, wherein the charge generation layer contains D type (Y type) titanyl phthalocyanine in which the maximum peak is provided at at least a Bragg angle 2θ±0.2° which is 27.2° and a peak is not provided at 26.2° in a CuKα characteristic X-ray diffraction spectrum, and a peak regarding a temperature change from 50° C. to 400° C., other than a peak by vaporization of absorption water is not provided in differential scanning calorimetry.
<12> The electrophotographic photoreceptor according to any one of the <1> to <11>, further comprising a blocking layer.

### Advantageous Effects of Invention

According to the present invention, an electrophotographic photoreceptor in which electrical characteristics by strong exposure fluctuate small, and mechanical characteristics and gas resistance are excellent is provided.

### BRIEF DESCRIPTION OF DRAWINGS

FIG. 1 is a schematic diagram illustrating an example of a main configuration of an image forming apparatus according to the present invention.

FIG. 2 is a CuKα characteristic X-ray diffraction spectrum of a charge generating material used in an example of the present invention.

### DESCRIPTION OF EMBODIMENTS

Hereinafter, an embodiment of the present invention will be described in detail. However, descriptions of configuration requirement which will be made below are just a representative example of the embodiment of the present invention, and the descriptions of configuration requirement may be appropriately changed and conducted in a range without departing from the gist of the present invention. In this specification, Me represents a methyl group, Et represents an ethyl group, and tBu represents a t-butyl group.

<<Electrophotographic Photoreceptor>>

A configuration of an electrophotographic photoreceptor according to the present invention will be described below. The electrophotographic photoreceptor according to the present invention is a laminate type electrophotographic photoreceptor in which a charge generation layer and a charge transport layer having a film thickness of 15 μm to 40 μm are provided on a conductive support (on a blocking layer in a case where the blocking layer such as an anodic oxide film or a resin layer is provided), and in which the charge transport layer is the outermost layer. The charge transport layer contains a charge transport material, a binder resin, an inorganic filler, and a compound represented by Formula (1), and may additionally contain an oxidant inhibitor, a leveling agent, and other additives, if necessary.

<Conductive Support>

The material of the conductive support is not particularly limited. The main examples thereof are as follows: a metal material such as aluminum, aluminum alloys, stainless steel, copper, and nickel; a resin material obtained by adding conductive powder particles of metal, carbon, tin oxide, and the like so as to impart conductivity; and a resin, glass, paper, and the like in which a conductive material such as aluminum, nickel, and indium oxide-tin oxide (ITO) is evaporated or applied onto the surface. The above materials may be singly used. A certain combination of two types or more at a certain proportion may be used.

Examples of the shape of the conductive support include a drum shape, a sheet shape, and a belt shape. Further, a support in which a conductive material having an appropriate resistance value is applied onto a conductive support formed of a metal material, in order to control conductivity or surface properties or to coat a defect may be used.

In a case where a metal material such as aluminum alloy is used as the conductive support, the conductive support may be coated with an anodic oxide film, and then may be used. In a case where coating with an anodic oxide film has been performed, a support subjected to sealing treatment by well-known methods is preferable.

The surface of the support may be smooth. The surface of the support may be roughened by using a special cutting method or by performing roughening treatment. In addition, roughening may be performed by mixing particles having an appropriate particle diameter, to a material constituting the support. In order to reduce price, a drawn pipe itself may be used without performing cutting treatment.

<Blocking Layer>

The electrophotographic photoreceptor according to the present invention may include a blocking layer. In a case where the blocking layer is to be applied, for example, the blocking layer may be formed in a manner that an oxide film is provided on the surface of a metal tube of the conductive support or a layer containing a resin component is provided on the surface of the metal tube or on the surface of the sheet-like photoreceptor, on which metal is evaporated.

In a case where the layer containing a resin component is applied, the layer may be only a resin or may further contain an inorganic filler. However, from a viewpoint that a layer having small humidity dependency is preferable, a resin layer in which metal oxide particles are contained as an inorganic filler is preferable.

As the metal oxide particle, a particle having high dispersion stability for a coating liquid is preferable. Specific examples of the metal oxide particle include silica, alumina, titanium oxide, barium titanate, zinc oxide, lead oxide, and indium oxide. Among the substances, a metal oxide particle showing n-type semiconductor characteristics is preferable. Titanium oxide, zinc oxide, or tin oxide is more preferable, and titanium oxide is particularly preferable.

Titanium oxide can be used in any form of being crystalline or amorphous. In a case of being crystalline, the crystalline type of titanium oxide may be any of an anatase type, a rutile type, and a brookite type. The anatase type or the rutile type is generally used because of water absorbency, efficiency of surface treatment, and the like. The rutile type may be particularly preferably used.

Regarding the particle diameter of the metal oxide particle, from a viewpoint of dispersion stability into a coating liquid, metal oxide particles having an average particle diameter which is equal to or less than 100 nm are generally preferable, and metal oxide particles having an average particle diameter of 10 to 60 nm are particularly preferable. The particle diameter of particles used in the coating liquid may be uniform or may be a combination of different particle diameters.

In a case of a combination of different particle diameters, particles having particle diameter distribution in which the maximum peak for the particle diameter is in the vicinity of 150 nm, and the minimum particle diameter is in a range from about 30 nm to about 500 nm are preferable. For example, particles having an average particle diameter of 0.1 μm and particles having an average particle diameter of 0.03 μm may be used in combination thereof.

Examples of a binder resin contained in the blocking layer include a resin material such as polyvinyl acetal, polyamide, phenol resin, polyester, epoxy resin, polyurethane, and polyacrylic acid. Among the substances, a polyamide resin which is excellent in adhesiveness to the support, and has small solubility for a solvent used in a charge generation layer coating liquid is preferable. Among the substances, polyamide which is also excellent in an aspect of handling and is usable in an alcohol solvent is more preferable.

Examples of such polyamide include commercial polyamide and an alcohol soluble copolymerized polyamide resin in which a diamine component represented by the following Structural formula 1 is provided as a constituent material. Examples of the commercial polyamide include ternary (6-66-610) or quaternary (6-66-610-12) copolymerized polyamide such as AMILAN CM4000 or CM8000 manufactured by Toray Corporation; methoxymethylated nylon resins such as TORESIN F-30K, MF-30, and EF-30T manufactured by Nagase ChemteX Corporation, and FINELEX FR-101, FR-104, FR-105, and FR-301 manufactured by Namariichi Co., Ltd.; polymerized fatty acid polyamide such as PA-100, PA-100A, PA-102A, PA-105A, PA-200, and PA-201 manufactured by T&K TOKA Corporation.; and polymerized fatty acid polyamide block copolymers such as TPAE-12 and TPAE-32 manufactured by T&K TOKA Corporation.

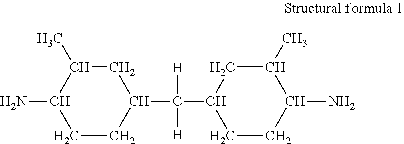 [SMILES: CC1CC(CC2CCC(N)C(C)C2)CCC1N]

Regarding the ratio between the metal oxide particles and the binder resin, the content of the metal oxide particles is generally equal to or more than 50 parts by mass and preferably equal to or more than 200 parts by mass, with respect to 100 parts by mass of the binder resin, from a viewpoint of electrical characteristics. From a viewpoint of stability and coating properties of the liquid, the content of the metal oxide particles is generally equal to or less than 800 parts by mass, and preferably equal to or less than 500 parts by mass.

Regarding the film thickness of the blocking layer, if the film thickness of the blocking layer is too thin, an effect for partial charging poorness is not sufficient, and if the film thickness of the blocking layer is too thick, an increase of a residual potential or degradation of adhesive strength between a conductive base and a photosensitive layer is caused.

The film thickness of the blocking layer is generally equal to or more than 0.1 μm, preferably equal to or more than 0.5 μm, and more preferably equal to or more than 1 μm. In addition, the film thickness of the blocking layer is generally equal to or less than 20 μm, preferably equal to or less than 10 μm, and more preferably equal to or less than 6 μm. The volume resistance value of the blocking layer is generally equal to or more than 1×10¹¹ Ω·cm, and preferably equal to or more than 1×10¹² Ω·cm. In addition, the volume resistance value of the blocking layer is generally equal to or less than 1×10¹⁴ Ω·cm, and preferably equal to or less than 1×10¹³ Ω·cm.

In order to obtain a blocking layer coating liquid which contains the metal oxide particles and the binder resin, the binder resin or a dissolution liquid obtained by dissolving the binder resin in an appropriate solvent may be mixed with slurry of metal oxide particles, which is ground by a planetary mill, a ball mill, a sand mill, a bead mill, a paint shaker, an attritor, or an ultrasonic wave or is treated in a dispersion treatment device. In addition, dissolving and stirring treatment may be performed. Conversely, metal oxide particles may be added to a binder resin dissolution liquid, and grinding or dispersion treatment may be performed in such a dispersion device.

<Charge Generation Layer>

A charge generation layer is formed by binding a charge generating substance to the binder resin. Examples of the charge generating substance include an inorganic photoconductive material such as selenium and alloys thereof, and cadmium sulfide, and an organic photoconductive material such as an organic pigment. Among the substances, the organic photoconductive material is preferable, and the organic pigment is particularly preferable.

Examples of the organic pigment include phthalocyanine pigments, azo pigments, dithioketopyrrolopyrrole pigments, squalene (squarylium) pigments, quinacridone pigments, indigo pigments, perylene pigments, polycyclic quinone pigments, anthanthrone pigments, and benzimidazole pigments. Among the pigments, the phthalocyanine pigment and the azo pigment are particularly preferable. In a case where an organic pigment is used as the charge generating substance, the organic pigment is used in a form of a dispersion layer in which fine particles of the organic pigment are bound to various binder resins.

In a case where a metal-free phthalocyanine compound, and a metal-containing phthalocyanine compound are used as the charge generating substance, a photoreceptor having high sensitivity to a laser beam having a relatively long wavelength, for example, to a laser beam having a wavelength in the vicinity of 780 nm is obtained. In a case where an azo pigment such as monoazo, diazo, and trisazo is used, a photoreceptor having sufficient sensitivity to white light, a laser beam having a wavelength in the vicinity of 660 nm, or a laser beam having a relatively short wavelength (for example, laser having a wavelength in the vicinity of 450 nm or 400 nm) can be obtained.

In a case where a phthalocyanine pigment is used as the charge generating substance, specific examples thereof include metal-free phthalocyanine; substances having crystal types of phthalocyanines in which metal such as copper, indium, gallium, tin, titanium, zinc, vanadium, silicon, germanium, and aluminum, oxide thereof, halide thereof, hydroxide thereof, alkoxide thereof, and the like are coordinated; and phthalocyanine dimers which use an oxygen atom as a crosslinking atom.

In particular, metal-free phthalocyanine of an X type or a τ type which is a crystal type having high sensitivity; titanyl phthalocyanine (another name: oxytitanium phthalocyanine) of an A type (another name: β type), a B type (another name: α type), a D type (another name: Y type), or the like; vanadyl phthalocyanine, chloroindium phthalocyanine, hydroxy indium phthalocyanine; chlorogallium phthalocyanine of a II type or the like; hydroxygallium phthalocyanine of a V type or the like; μ-oxo-gallium phthalocyanine dimers of a G type, an I type, or the like; or μ-oxo-aluminum phthalocyanine dimers of a II type or the like is preferable.

Among these types of phthalocyanine, titanyl phthalocyanine of the A type (another name: β type), the B type (another name: α type), and the D type (another name: Y type) in which a clear peak is shown at a diffraction angle 2θ(±0.2°) in powder X-ray diffraction, which is 27.1°, 27.2°, or 27.3°; the II type chlorogallium phthalocyanine; hydroxygallium phthalocyanine which has the V type, has a strongest peak at 28.1°, has a clear peak at 28.1° without a peak at 26.2°, and has a half value width W at 25.9°, which satisfies 0.1°≦W≦0.4°; the G type μ-oxo-gallium phthalocyanine dimers, and the like are more preferable. From a viewpoint of stability of sensitivity and electrical characteristics, a D type (Y type) titanyl phthalocyanine which has the maximum peak at at least a Bragg angle 2θ±0.2° of 27.2° and does not have a peak at 26.2° in a CuKα characteristic X-ray diffraction spectrum, and which does not have a peak in a change of a temperature from 50° C. to 400° C., other than a peak by vaporization of absorption water in differential scanning calorimetry is further preferable.

The phthalocyanine compounds may be singly used or may be used in a mixture or in a mixed crystalline state of some compounds. Here, as a mixed state in which the phthalocyanine compound and the like are in a crystalline state, a mixture obtained by mixing the components later may be used or the mixed state may be caused in a manufacturing and treatment process of a phthalocyanine compound, such as synthesis, pigmentation, and crystallization. As such treatment, acid paste treatment, grinding treatment, solvent treatment, and the like are known. In order to cause the mixed crystalline state, as disclosed in JP-A-10-48859, a method in which, after two types of crystals are mixed, the mixture is mechanically ground so as to perform amorphizing, and then solvent treatment is performed to perform conversion to a specific crystalline state is exemplified.

In a case where the azo pigment is used as the charge generating substance, various bisazo pigments and trisazo pigments are suitably used. In a case where the organic pigment is used as the charge generating substance, the organic pigment may be singly used or may be used in combination of two types or more of pigments. In this case, a combination of two types or more of charge generating substances which have spectral sensitivity characteristics in different spectral regions, that is, a visible region and a near-infrared region is preferably used. Among the pigments, a combination of a disazo pigment or a triazo pigment with a phthalocyanine pigment is more preferably used.

The binder resin used in the charge generation layer is not particularly limited. Examples of the binder resin include polyvinyl butyral resins, polyvinyl formal resins, polyvinyl acetal resins (such as partially-acetalized polyvinyl butyral resins in which a portion of butyral is modified with formal or acetal), polyarylate resins, polycarbonate resins, polyester resins, modified ether polyester resins, phenoxy resins, polyvinyl chloride resins, polyvinylidene chloride resins, polyvinyl acetate resins, polystyrene resins, acrylic resins, methacrylic resins, polyacrylamide resins, polyamide resins, polyvinyl pyridine resins, cellulose resins, polyurethane resins, epoxy resins, silicone resins, polyvinyl alcohol resins, polyvinyl pyrrolidone resins, caseins, vinyl chloride-vinyl acetate copolymers (such as vinyl chloride-vinyl acetate copolymers, hydroxy-modified vinyl chloride-vinyl acetate copolymers, carboxyl-modified vinyl chloride-vinyl acetate copolymers, and vinyl chloride-vinyl acetate-maleic anhydride copolymers), styrene-butadiene copolymers, vinylidene chloride-acrylonitrile copolymers, insulating resins (such as styrene-alkyd resins, silicon-alkyd resins, and phenol-formaldehyde resins), and organic photoconductive polymers (such as poly-N-vinylcarbazole, polyvinylanthracene, and polyvinylperylene). The binder resins may be singly used or may be used in mixture obtained at a certain combination of two types or more thereof.

The charge generation layer is formed, specifically, for example, in a manner that a charge generating substance is dispersed in a solution in which the above-described binder resin is dissolved in an organic solvent, so as to prepare a coating liquid, and the prepared coating liquid is applied onto a conductive support (onto a blocking layer in a case where the blocking layer is provided).

Regarding the mixing ratio (mass) between the binder resin and the charge generating substance in the charge generation layer, the content of the charge generating substance is in a range of being generally equal to or more than 10 parts by mass, preferably equal to or more than 30 parts by mass, with respect to 100 parts by mass of the binder resin. In addition, the content thereof is in a range of being generally equal to or less than 1000 parts by mass, and preferably equal to or less than 500 parts by mass. The film thickness thereof is in a range of being generally equal to or more than 0.1 μm, and preferably equal to or more than 0.15 μm. In addition, the film thickness thereof is in a range of being generally equal to or less than 10 μm, and preferably equal to or less than 0.6 μm. If the percentage of the charge generating substance is too high, stability of the coating liquid may be deteriorated by an aggregation of the charge generating substance, and the like. If the percentage of the charge generating substance is too low, degradation of sensitivity as a photoreceptor may be caused.

As a method of dispersing the charge generating substance, for example, well-known dispersing methods such as a ball mill dispersing method, an attritor dispersing method, a sand mill dispersing method, a bead mill dispersing method are exemplified. At this time, it is effective that the size of particles is reduced so as to have a particle size in a range of being generally equal to or less than 0.5 μm and preferably equal to or less than 0.3 μm, and more preferably equal to or less than 0.15 μm.

<Charge Transport Layer>

The charge transport layer in the present invention may be obtained in a manner that an inorganic filler and a hydrocarbon compound represented by Formula (1) in addition to a charge transport material and the like, a binder resin are dissolved, dispersed, or mixed so as to manufacture a coating liquid, the obtained coating liquid is applied onto the charge generation layer, and then drying is performed.

It is preferable that the film thickness of the charge transport layer is equal to or more than 15 μm from a viewpoint of electrical characteristics, and is equal to or more than 17 μm from a viewpoint of strong-exposure resistance characteristics. From a viewpoint of image stability and a resolution, the film thickness thereof is equal to or less than 40 μm and preferably equal to or less than 35 μm.

It is considered that the reason is as follows. The inorganic filler which is non-compatible in a photosensitive layer does not relate to charge transfer, and the presence of the inorganic filler causes a charge transfer path to be restricted. In addition, the electrical characteristics of a photoreceptor function as an important control factor for causing the film thickness of the photosensitive layer to rule sensitivity and a residual potential. As a result obtained by the balance relationship thereof, preferable strong-exposure resistance characteristics are imparted in the above range of the film thickness.

Known additives such as a plasticizer, a lubricant, a dispersion auxiliary agent, an oxidant inhibitor, an ultraviolet absorbing agent, an electron attracting compound, a dye, a pigment, a sensitizer, a leveling agent, a stabilizing agent, a fluidity imparting agent, and a crosslinking agent may be contained in order to improve film forming properties, flexibility, coating properties, stain resistance, gas resistance, light resistance, and the like of the charge transport layer, or in order to further improve mechanical strength of the photosensitive layer.

Examples of the oxidant inhibitor include a hindered phenol compound and hindered amine compound. Examples of the dye or the pigment include various coloring compounds and various azo compounds. Examples of the leveling agent include silicone oil and a fluorochemical surfactant.

[Inorganic Filler]

Examples of the inorganic filler used in the present invention include oxide, nitride or composite oxide of typical elements and transition elements, such as titanium oxide, silicon oxide (silica), tin oxide, aluminum oxide (alumina), zirconium oxide, indium oxide, silicon nitride, calcium oxide, zinc oxide, barium sulfate, and barium titanate. Among the substances, silica is preferable from a viewpoint of dispersion efficiency in a coating liquid, and electrical characteristics.

Silica is manufactured by a vapor phase method or a liquid phase method, and silica of which the surface is subjected to surface modification by a reactive organosilicon compound is preferable. The silica subjected to surface modification may be manufactured by a dry type method and a wet type method.

In the dry type method, manufacturing may be performed in a manner that a surface treatment agent is mixed with metal oxide particles so as to coat the metal oxide particles with the surface treatment agent, and, if necessary, heating treatment is performed. In the wet type method, manufacturing may be performed in a manner that metal oxide particles and a mixture obtained by mixing a surface treatment agent to an appropriate solvent are stirred well until being uniformly adhered to each other or in a manner that the metal oxide particles and the mixture are mixed in a medium, and then drying and, if necessary, heating treatment is performed.

Examples of the reactive organosilicon compound include a silane coupling agent, a silane treatment agent, and a siloxane compound. Among the substances, the silane treatment agent is preferable from a viewpoint of reactivity with a particulate organosilicon compound, and from a viewpoint of suppressing generation of a reactive aggregate particle at which a not-reactive portion easily remains.

Among silane treatment agents, a silane treatment agent having an alkyl group which has 1 to 3 carbon atoms is preferable. Examples of such a silane treatment agent include hexamethyl disilazane, trimethyl methoxysilane, trimethyl ethoxysilane, trimethyl chlorosilane, dimethyl dichlorosilane, dimethyl dimethoxysilane, dimethyl ethoxysilane, methyl dimethoxysilane, methyl trimethoxysilane, and methyl triethoxysilane.

Sphericity of the silica is generally equal to or higher than 0.95, preferably equal to or higher than 0.96, and more preferably equal to or higher than 0.98, from a viewpoint of crack resistance. As the sphericity is increased, the surface area of silica is reduced, and an interface functioning as a cause of an occurrence of cracks is reduced. Thus, the occurrence of cracks is difficult. Density of the silica is generally equal to or more than 1.5 g/cm³, preferably equal to or more than 1.8 g/cm³, and more preferably equal to or more than 2.0 g/cm³, from a viewpoint of suppressing the occurrence of cracks, and from a viewpoint of crack resistance. The density thereof is generally equal to or less than 3.0 g/cm³, preferably equal to or less than 2.8 g/cm³, and more preferably equal to or less than 2.5 g/cm³.

The average primary particle diameter of the inorganic filler is preferably equal to or less than 1.0 μm, and more preferably equal to or less than 0.8 μm, from a viewpoint of coating liquid stability. From a viewpoint of abrasion resistance, the average primary particle diameter thereof is preferably equal to or more than 0.01 μm. From a viewpoint of filming resistance, the average primary particle diameter thereof is more preferably equal to or more than 0.1 μm and further preferably equal to or more than 0.3 μm. The average primary particle diameter may be obtained by measuring of a scanning electron microscope (SEM) or a transmission electron microscope (TEM).

The content of the inorganic filler is preferably equal to or more than 5 mass %, with respect to the binder resin in the charge transport layer. The content of the inorganic filler is more preferably equal to or more than 6 mass %, from a viewpoint of filming resistance. The content of the inorganic filler is preferably equal to or less than 30 mass %, in order to ensure intensity as strong as the photoreceptor is not soft. The content of the inorganic filler is more preferably equal to or less than 25 mass %, from a viewpoint of dispersibility in the charge transport layer and the electrical characteristics.

[Hydrocarbon Compound]

In the present invention, the charge transport layer contains a hydrocarbon compound represented by Formula (1). The percentage of the hydrocarbon compound which is represented by Formula (1) and is contained in the charge transport layer is preferably equal to or more than 10 mass %, more preferably equal to or more than 20 mass %, with respect to the inorganic filler from a viewpoint of dispersibility of the inorganic filler in a photosensitive layer. From a viewpoint of mechanical properties of the photosensitive layer, the percentage thereof is preferably equal to or less than 100 mass %, and more preferably equal to or less than 80 mass %.

[Charge Transport Material]

The charge transport material used in the present invention includes well-known charge transport materials which are generally used. The charge transport materials may be singly used or may be used in combination of two types or more thereof. The percentage of the charge transport material to be used, to the binder resin used in the charge transport layer is generally equal to or more than 30 parts by weight, preferably equal to or more than 35 parts by weight, more preferably equal to or more than 40 parts by weight, with respect to 100 parts by weight of the binder resin from a viewpoint of the electrical characteristics. From a viewpoint of abrasion resistance, the percentage thereof is generally equal to or less than 100 parts by weight, preferably equal to or less than 90 parts by weight, and more preferably equal to or less than 85 parts by weight.

A structure of a charge transport material suitable for the present invention will be described below as examples. The following structures are just examples for specifically describing the present invention, and it is not limited to the following structures in a range without departing from the concept of the present invention.

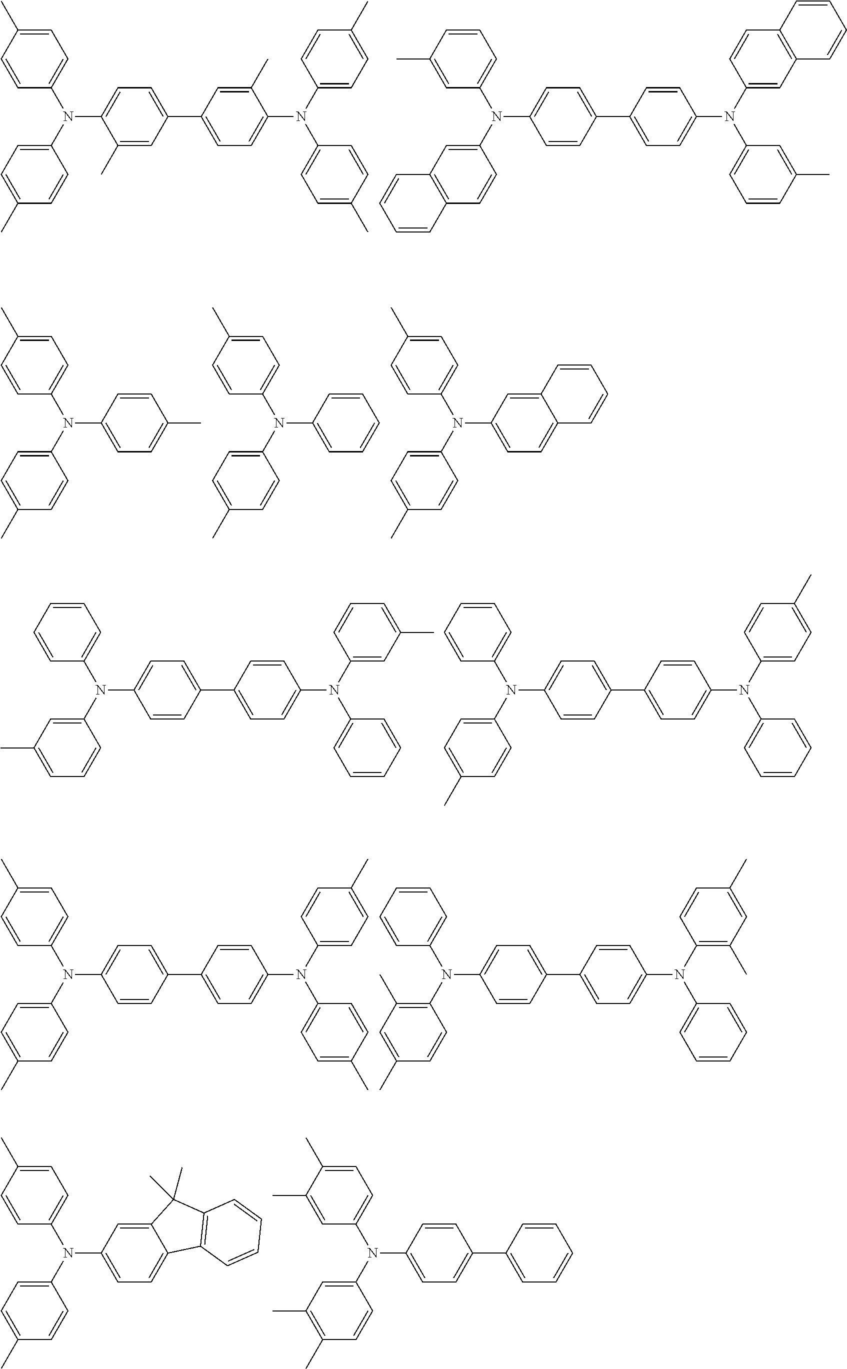

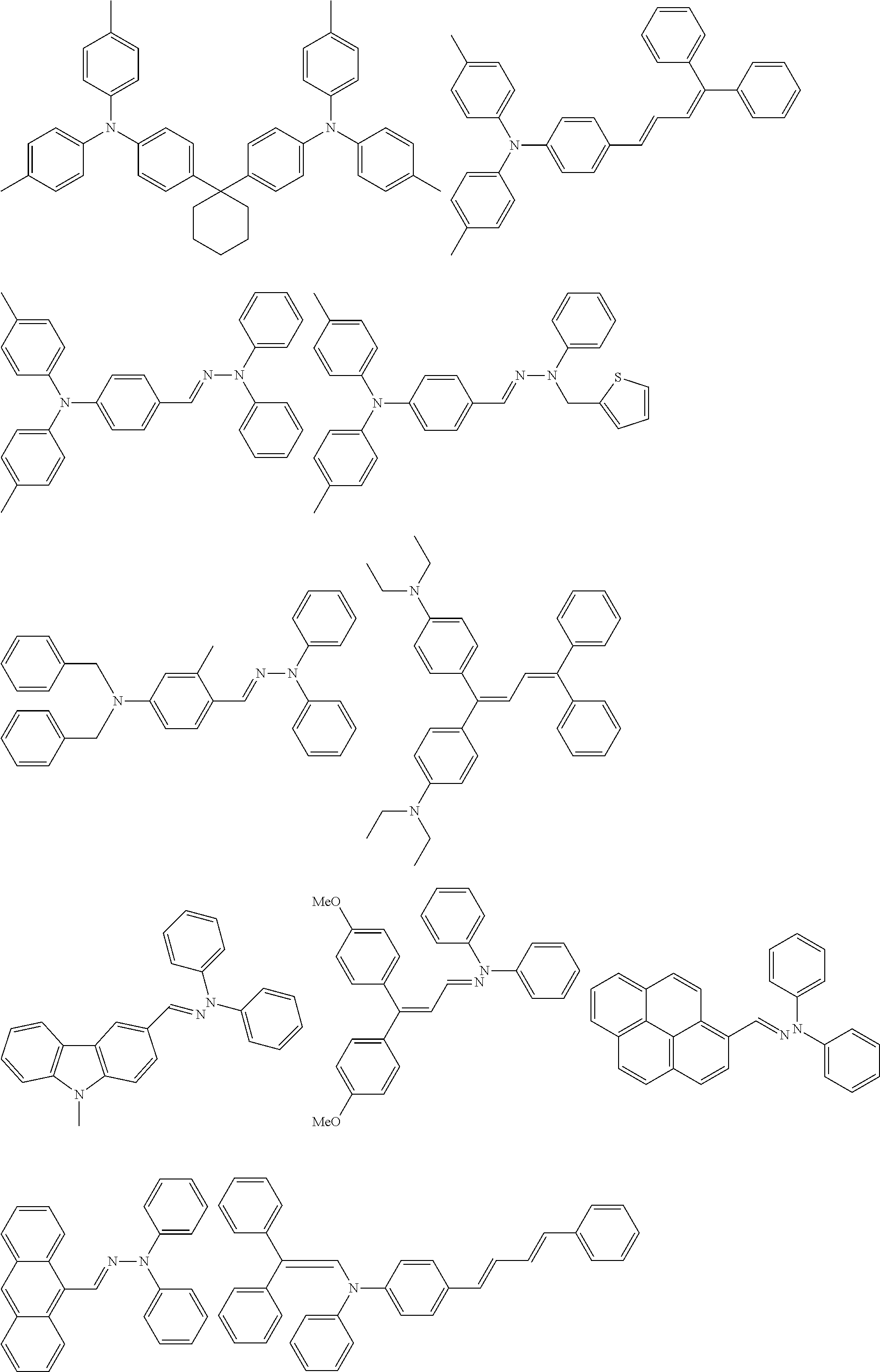

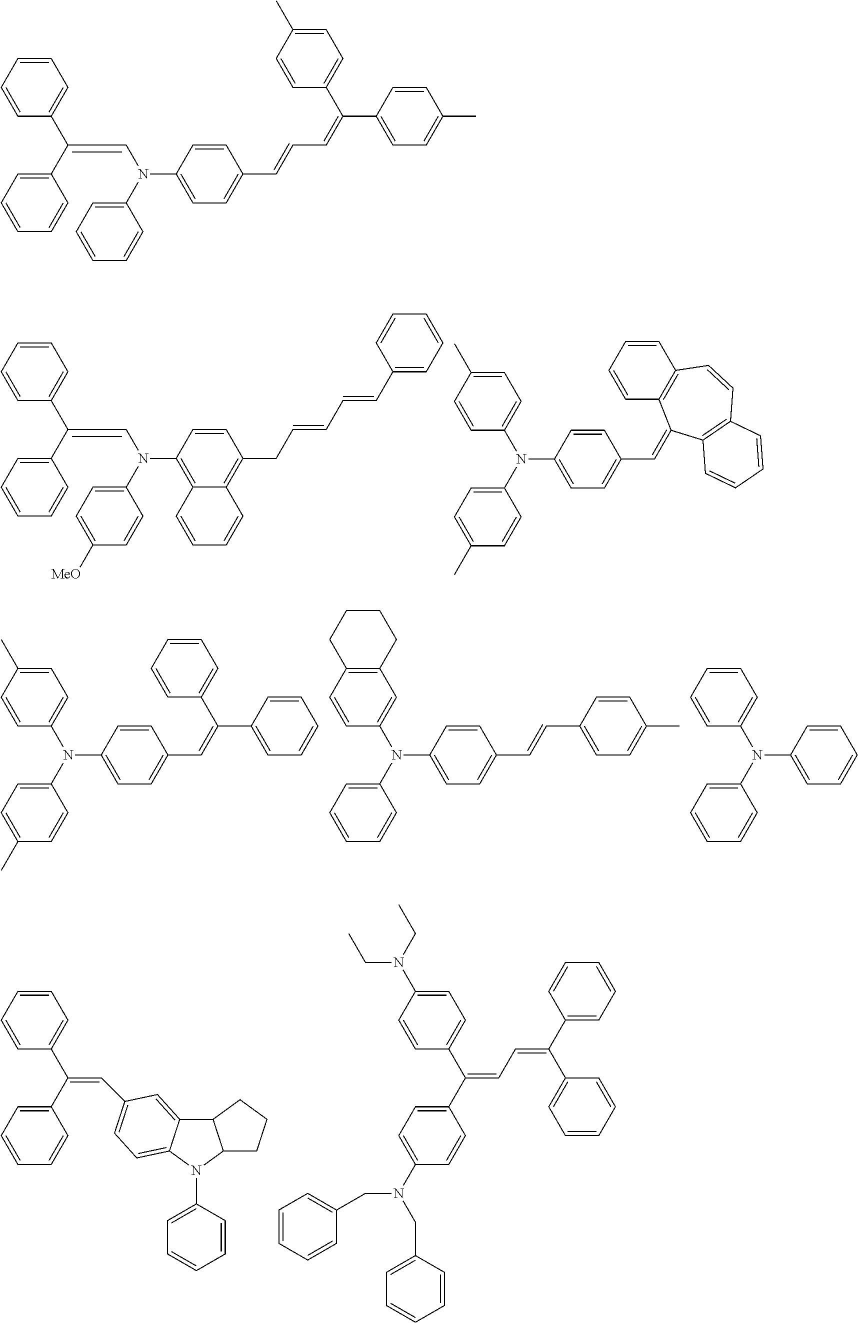

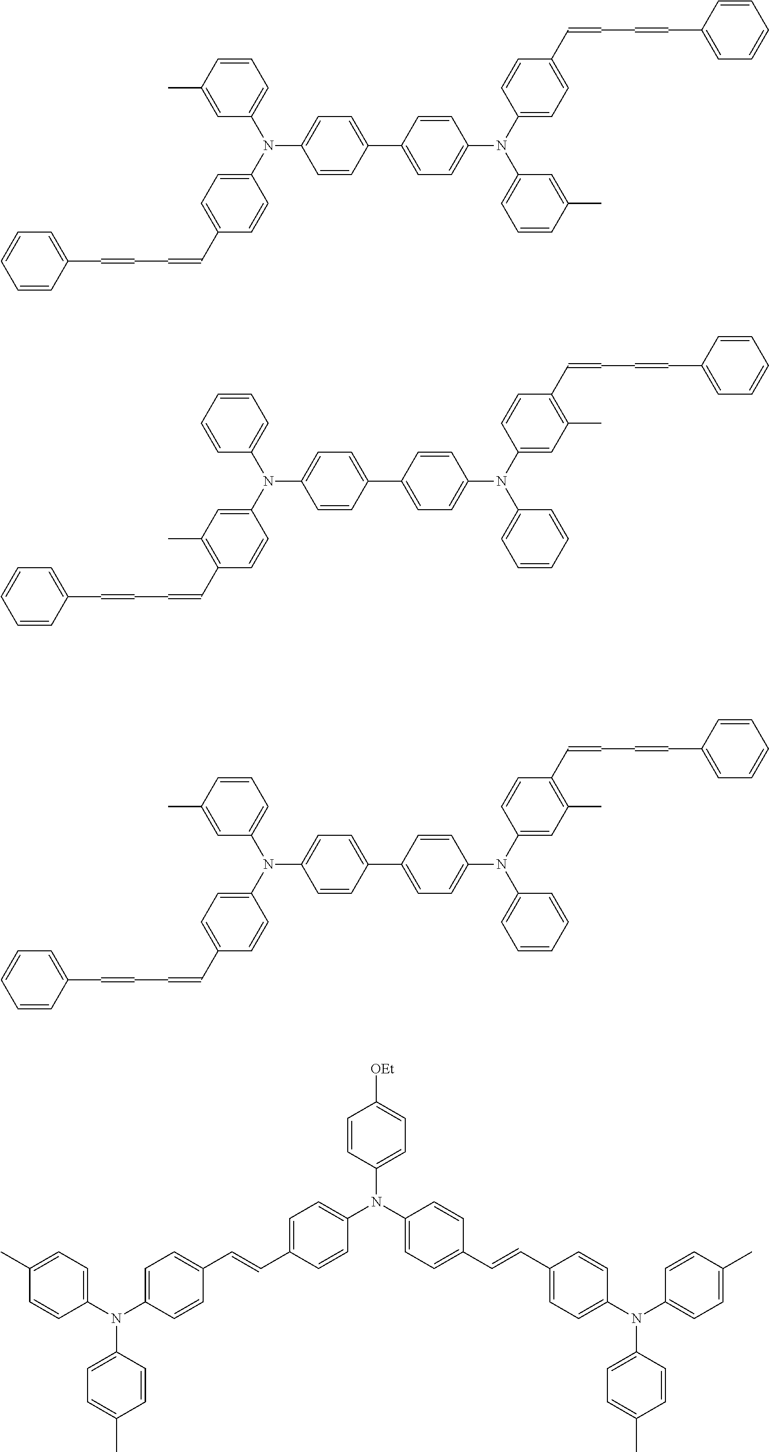

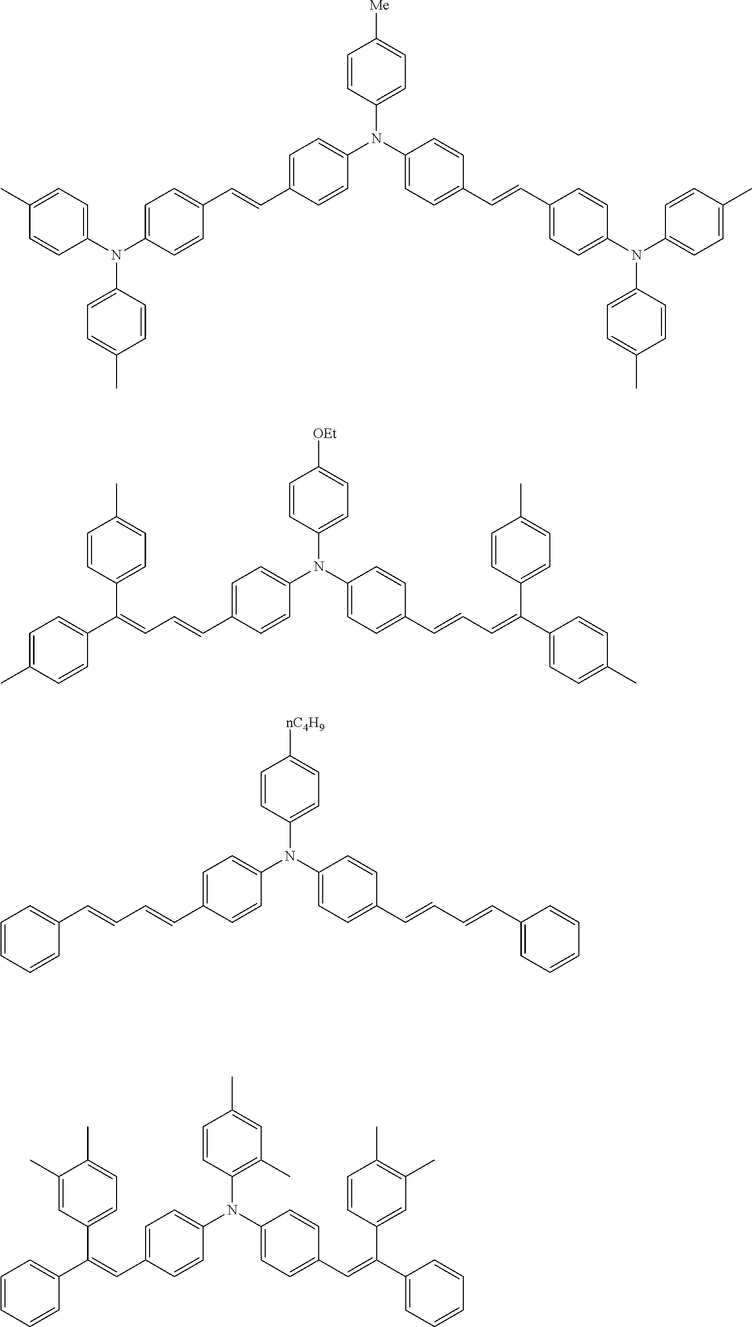

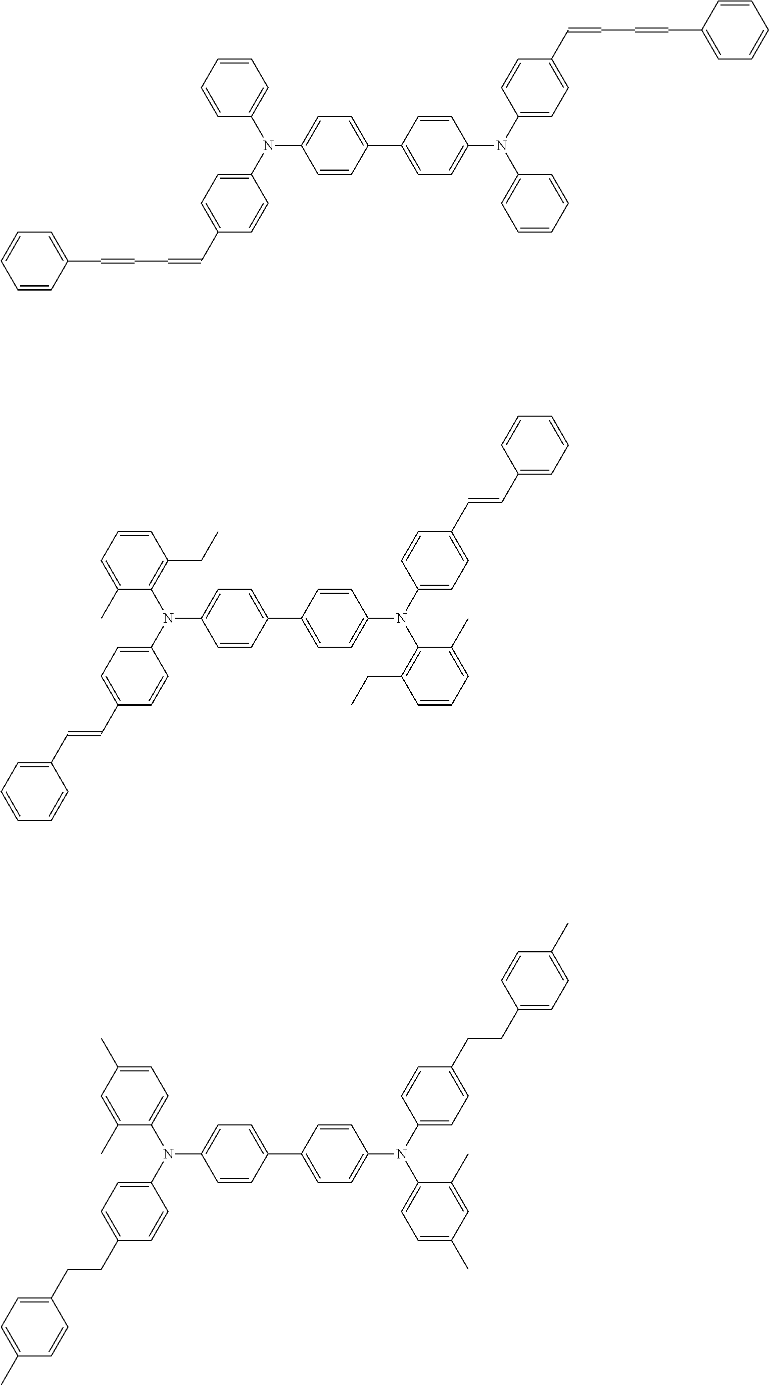 [SMILES: C(C=Cc1ccc(N(c2ccccc2)c2ccc(-c3ccc(N(c4ccccc4)c4ccc(C=CC=Cc5ccccc5)cc4)cc3)cc2)cc1)=Cc1ccccc1 ; CCc1cccc(C)c1N(c1ccc(/C=C/c2ccccc2)cc1)c1ccc(-c2ccc(N(c3ccc(/C=C/c4ccccc4)cc3)c3c(C)cccc3CC)cc2)cc1 ; Cc1ccc(CCc2ccc(N(c3ccc(-c4ccc(N(c5ccc(CCc6ccc(C)cc6)cc5)c5ccc(C)cc5C)cc4)cc3)c3ccc(C)cc3C)cc2)cc1]

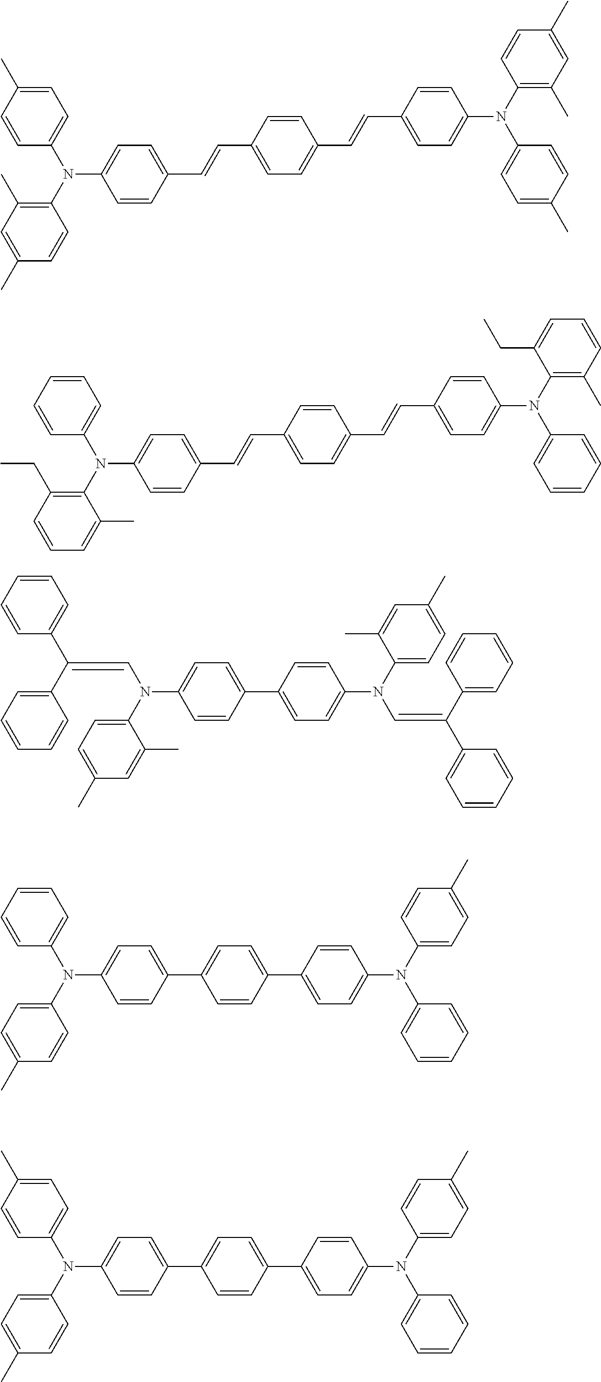

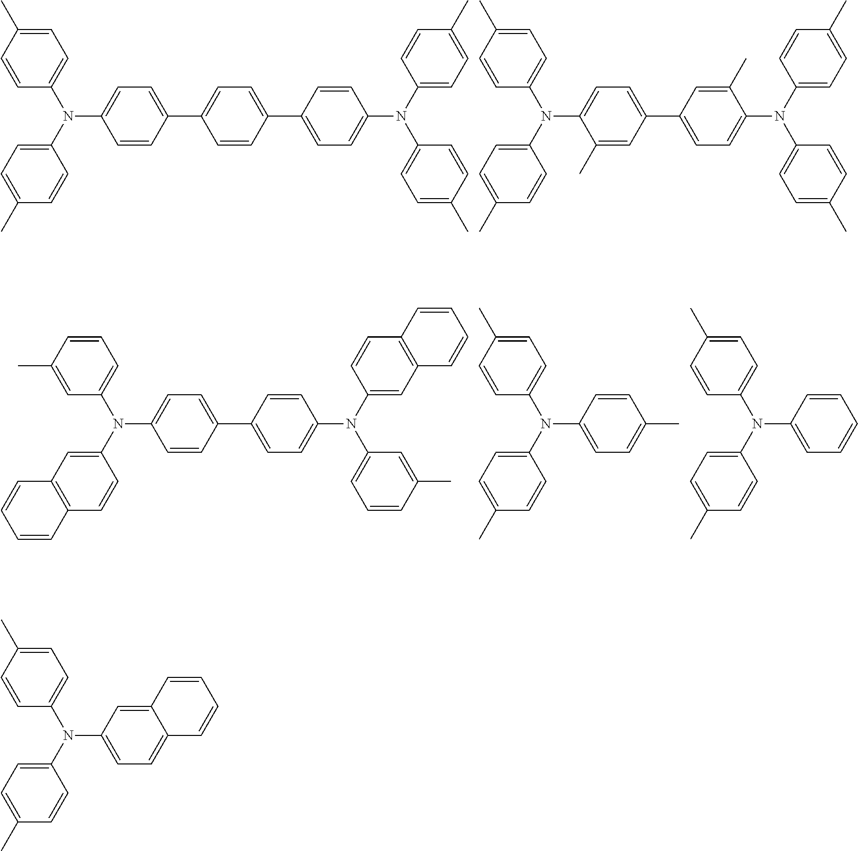

[Binder Resin]

The charge transport layer is formed in a form in which the above-described inorganic filler, the hydrocarbon compound represented by Formula (1), and the charge transport material are bound to a binder resin. Examples of the binder resin used in the charge transport layer include polymethyl methacrylate, polystyrene, vinyl polymers such as polyvinyl chloride, and copolymers thereof, polycarbonate, polyarylate, polyester, polyester carbonate, polysulfone, polyimide, phenoxy resin, epoxy resin, and silicone resins. In addition, partically-crosslinked cured matters of the above substances may be also used. Among the binder resins, from a viewpoint of electrical characteristics of a photoreceptor, a polycarbonate resin, or a polyarylate resin is particularly preferable. The above resins may be singly or may be used in mixture of plural types thereof.

Specific examples of a suitable structure of the binder resin will be described below. The specific examples are just described for exemplification, and a mixture with a certain well-known binder resin may be used in a range without departing from the purpose of the present invention.

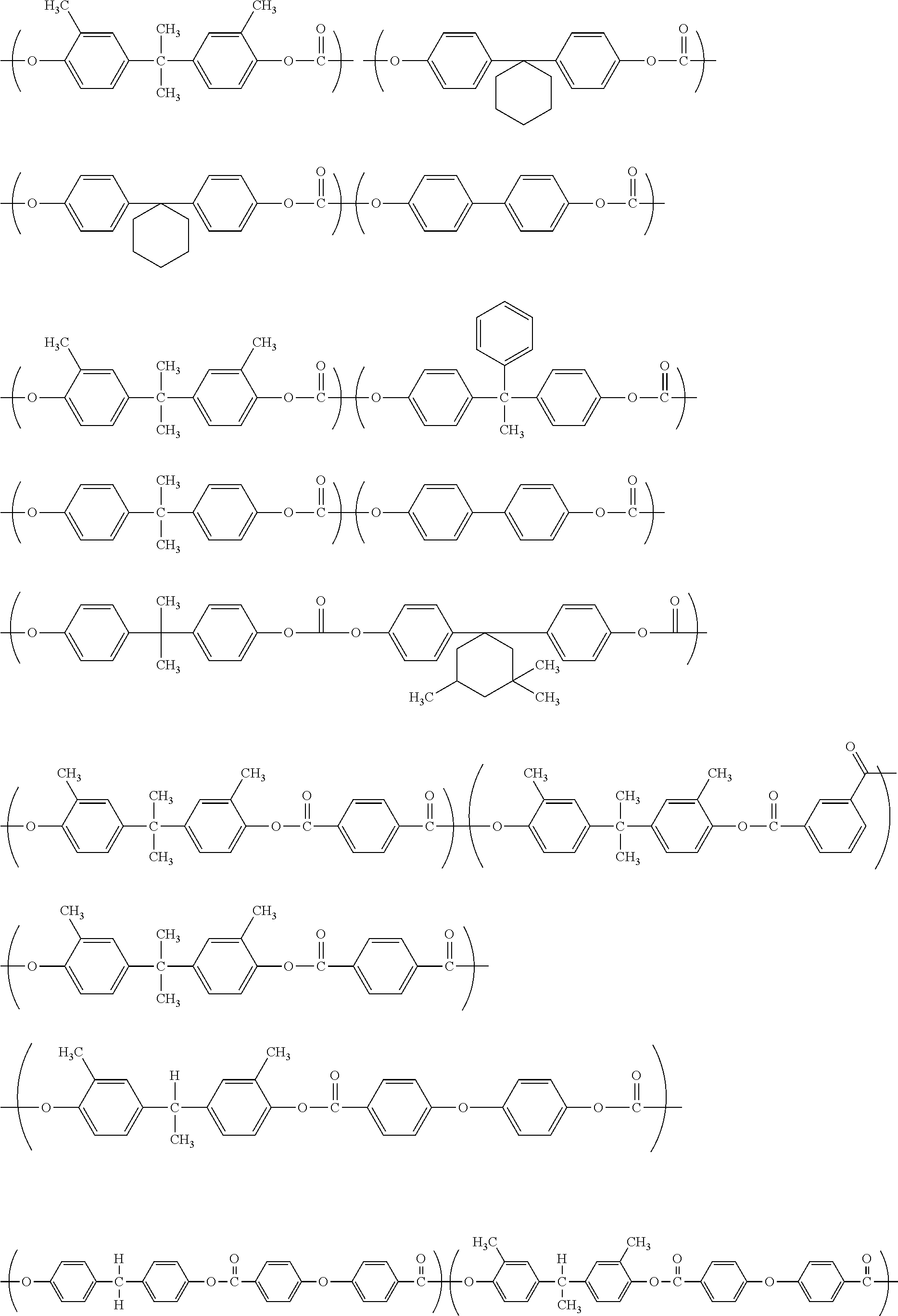

The viscosity-average molecular weight (Mv) is generally equal to or more than 20,000, preferably equal to or more than 30,000, and further preferably equal to or more than 40,000. The viscosity-average molecular weight is generally equal to or less than 200,000, preferably equal to or less than 100,000, and further preferably equal to or less than 80,000.

In a case where the viscosity-average molecular weight (Mv) is excessively small, mechanical strength at a time of formation as a film, for example, for forming a photoreceptor tends to be degraded. In a case where the viscosity-average molecular weight (Mv) is excessively large, viscosity as a coating liquid is increased, and thus a tendency that coating so as to have an appropriate film thickness is difficult occurs, and dispersibility of the inorganic filler may be deteriorated.

[Electron Attracting Compound]

In the present invention, an electron attracting compound may be used for improving an effect of suppressing optical fatigue. Examples of the electron attracting compound include a cyano compound of aromatic esters having tetracyano quinodimethane, dicyano quinomethane, or a dicyano quinovinyl group, a nitro compound such as 2,4,6-trinitrofluorenone, a condensed polycyclic aromatic compound such as perylene, diphenoquinone derivatives, quinones, aldehydes, ketones, esters, acid anhydrides, phthalides, a metal complex of substituted and unsubstituted salicylic acid, a metal salt of substituted and unsubstituted salicylic acid, a metal complex of aromatic carboxylic acid, and a metal salt of aromatic carboxylic acid. Among the compounds, from a viewpoint of the electrical characteristics, diphenoquinone derivatives and quinone derivatives as represented by Formula (2) are preferable.

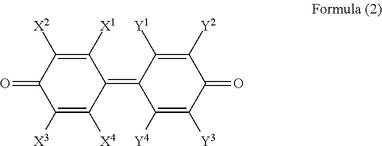

X¹, X², X³, X⁴, Y¹, Y², Y³, and Y⁴ in Formula (2) each respectively indicate a hydrogen atom, an alkyl group, an aryl group, an acyl group, or a bivalent organic group. A ring structure between X¹ and X², a ring structure between X³ and X⁴, a ring structure between Y¹ and Y², and a ring structure between Y³ and Y⁴ may be provided.

Examples of the alkyl group include a straight-chain alkyl group such as a methyl group, an ethyl group, an n-propyl group, an n-butyl group, an n-hexyl group, and an n-octyl group; a branched alkyl group such as an i-propyl group, an ethylhexyl group, and a t-butyl group; and a cyclic alkyl group such as a cyclohexyl group. Among the groups, from a viewpoint of the electrical characteristics, a straight-chain alkyl group or a branched alkyl group is preferable. Examples of the aryl group include a phenyl group, a naphthyl group, a biphenyl group, an anthryl group, a phenanthryl group, a tolyl group, and an anisyl group. Among the groups, from a viewpoint of the electrical characteristics, a phenyl group is preferable. Compounds represented by Formula (2) will be exemplified below.

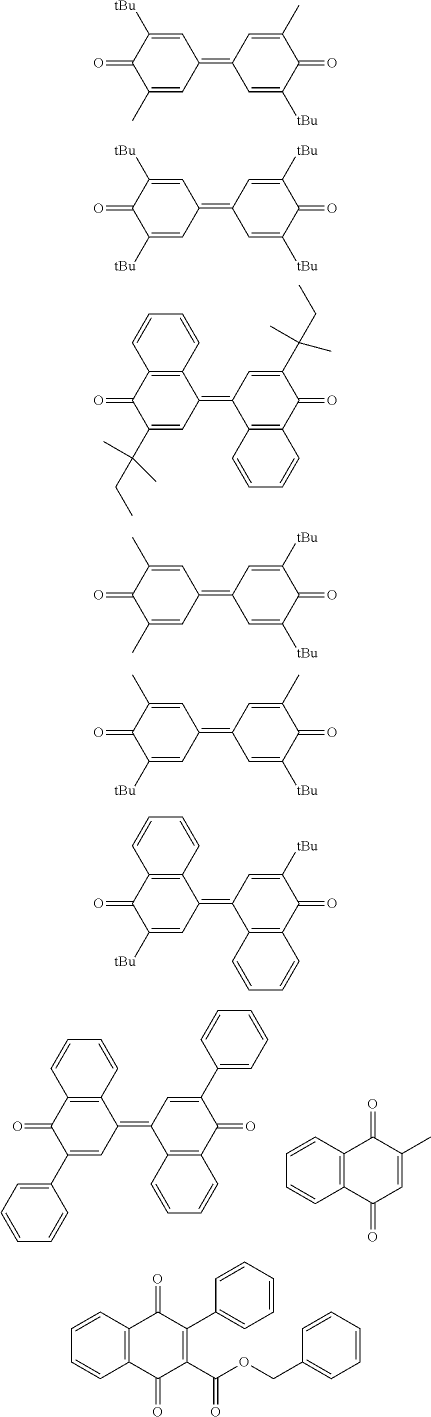

Among the compounds, the following structure having an alkyl substituent is preferably used because of being easily obtained.

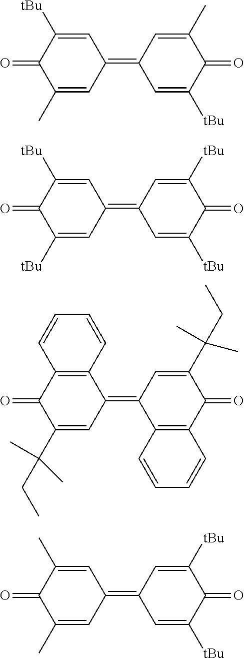

The content of the electron attracting compound is generally equal to or more than 2 mass %, preferably equal to or more than 4 mass %, with respect to the inorganic filler contained in the charge transport layer from a viewpoint of strong-exposure resistance characteristics. From a viewpoint of chargeability and a surface potential, the content thereof is generally equal to or less than 50 mass %, and preferably equal to or less than 40 mass %.

[Organic Solvent]

As an organic solvent used in a coating liquid for forming a charge transport layer, for example, the followings may be used: ethers such as tetrahydrofuran, 1,4-dioxane, and dimethoxyethane; esters such as methyl formate and ethyl acetate; ketones such as acetone, methyl ethyl ketone, and cyclohexanone; aromatic hydrocarbons such as benzene, toluene, and xylene; chlorinated hydrocarbons such as dichloromethane, chloroform, 1,2-dichloroethane, 1,1,2-trichloroethane, 1,1,1-trichloroethane, tetrachloroethane, 1,2-dichloropropane, and trichloroethylene; nitrogen-containing compounds such as n-butylamine, isopropanolamine, diethylamine, triethanolamine, ethylenediamine, and triethylenediamine; and aprotic polar solvents such as acetonitrile, N-methylpyrrolidone, N,N-dimethylformamide, and dimethylsulfoxide.

Among the solvents, from a viewpoint of suppressing brushing, an organic solvent which contains ether having a boiling point of 90° C. or lower as the main component, and contains 5 mass % to 50 mass % of ether having a boiling point of 120° C. or higher is preferable. As the ether having a boiling point of 90° C. or lower, from a viewpoint of brushing resistance and safety, ether having a boiling point of 50° C. or higher is preferable, and ether having a boiling point of 60° C. or higher is more preferable.

Examples of such ether include tetrahydrofuran, dimethoxyethane, dioxolane, methyltetrahydrofuran, and tetrahydropyran. Among the substances, from a viewpoint of solubility and the like of the binder resin, cyclic ether is preferable and tetrahydrofuran is particularly preferable.

The content of ether having a boiling point of 90° C. or lower is preferably equal to or more than 50 mass % in the total organic solvent. The content thereof is more preferably equal to or more than 60 mass %, and further preferably equal to or more than 75 mass %, from a viewpoint of a dry rate of a coating film. From a viewpoint of brushing resistance, the content thereof is preferably equal to or less than 90 mass %, and more preferably equal to or less than 85 mass %.

As ether having a boiling point of 120° C. or higher, from a viewpoint of a dry rate and a residual solvent, ether having a boiling point of 200° C. or lower is preferable, and ether having a boiling point of 170° C. or lower is more preferable. Examples of such ether include diethoxyethane, anisole, and 2,2-ditetrahydrofurylpropane. Aromatic ether is preferable, and anisole is particularly preferable.

The content of the ether having a boiling point of 120° C. or higher is preferably equal to or more than 10 mass % and is more preferably equal to or more than 15 mass %, in the total organic solvent from a viewpoint of brushing resistance. From a viewpoint of a dry rate of a coating film, the content thereof is preferably equal to or less than 30 mass %, and more preferably equal to or less than 25 mass %.

In addition, to ether having a boiling point of 90° C. or lower and ether having a boiling point of 120° C. or higher, a certain organic solvent may be mixed in a range in which the binder resin is not precipitated. Examples of such an organic solvent include ether having a boiling point of 90° C. to 120° C., ketone such as methyl ethyl ketone, and alcohol having 4 or more carbon atoms. The content of the organic solvent is preferably 60 to 95 mass %, more preferably 70 to 90 mass %, and particularly preferably 75 to 85 mass %, with respect to the entirety of the coating liquid.

<Forming Method of Each Layer>

Each layer constituting a photoreceptor is formed by sequentially repeating a coating and dry process for each layer. The coating and dry process is performed by well-known methods such as dip coating, spray coating, nozzle coating, a bar coater, a roll coater, and blade coating. The above coating with a coating liquid is performed on a support, and the coating liquid is obtained in a manner that substances to be contained in a layer are dissolved or dispersed in a solvent.

A solvent or a dispersion medium to be used is not particularly limited. However, specific examples thereof include ethers such as tetrahydrofuran, 1,4-dioxane, and dimethoxyethane; esters such as methyl formate and ethyl acetate; ketones such as acetone, methyl ethyl ketone, and cyclohexanone; aromatic hydrocarbons such as benzene, toluene, and xylene; chlorinated hydrocarbons such as dichloromethane, chloroform, 1,2-dichloroethane, 1,1,2-trichloroethane, 1,1,1-trichloroethane, tetrachloroethane, 1,2-dichloropropane, and trichloroethylene; nitrogen-containing compounds such as n-butylamine, isopropanolamine, diethylamine, triethanolamine, ethylenediamine, and triethylenediamine; and aprotic polar solvents such as acetonitrile, N-methylpyrrolidone, N,N-dimethylformamide, and dimethylsulfoxide. The above substances may be singly used or may be used in a certain combination of two types or more and different types may be used together.

The amount of the used solvent or dispersion medium is not particularly limited. However, considering the purpose of each layer and properties of a selected solvent or dispersion medium, it is preferable that the amount thereof is appropriately adjusted to cause physical properties such as solid concentration or viscosity of the coating liquid to be in a desired range.

In a case of the charge transport layer, the solid concentration of a coating liquid is set to be in a range of being generally equal to or more than 5 mass %, and preferably equal to or more than 10 mass %, and to be in a range of being generally equal to or less than 40 mass %, and preferably equal to or less than 35 mass %. The viscosity of the coating liquid is set to be in a range of being generally equal to or more than 10 cps and preferably equal to or more than 50 cps, and to be in a range of being generally equal to or less than 500 cps and preferably equal to or less than 400 cps.

In a case of the charge generation layer, the solid concentration of a coating liquid is set to be in a range of being generally equal to or more than 0.1 mass %, and preferably equal to or more than 1 mass %, and to be in a range of being generally equal to or less than 15 mass %, and preferably equal to or less than 10 mass %. The viscosity of the coating liquid is set to be in a range of being generally equal to or more than 0.01 cps and preferably equal to or more than 0.1 cps, and to be in a range of being generally equal to or less than 20 cps and preferably equal to or less than 10 cps.

As a coating method with a coating liquid, for example, a dip coating method, a spray coating method, a spinner coating method, a bead coating method, a wire-bar coating method, a blade coating method, a roller coating method, an air-knife coating method, a curtain coating method, and the like are exemplified. Other well-known coating methods may be also used.

<<Image Forming Apparatus>>

An image forming apparatus such as a copier and printer, which uses an electrophotographic photoreceptor according to the present invention includes processes of at least charging, exposure, developing, transfer, and cleaning, and any process may be performed by using any of methods which are generally used.

As a charging method (charging machine), for example, direct charging means for bringing a direct charging member to which a voltage is applied, into contact with the surface of a photoreceptor so as to perform charging may be used in addition to corotron using corona discharge, and scorotron charging. As the direct charging means, means using any contact charging and the like by a conductive roller, or a brush, a film, or the like may be used. Any of means according to aerial discharge, and means for injection charging which does not accord with the aerial discharge may be used. Among the above means, in a charging method using corona discharge, scorotron discharge is preferable for holding a dart portion potential to be uniform. As a charging type in a case of a contact charging device using a conductive roller and the like, DC charging or AC-superimposed DC charging may be used.

As exposure light, for example, a halogen lamp, a fluorescent lamp, a laser (semiconductor, and He—Ne), an LED, an in-photoreceptor exposure type, and the like are exemplified. However, as a digital electrophotographic type, a laser, an LED, an optical shutter array, and the like are preferably used. Regarding a wavelength, monochromatic light having a slightly-short wavelength tendency in a region of 600 to 700 nm, and monochromatic light having a short wavelength in a region of 380 to 500 nm may be used in addition to monochromatic light of 780 nm.

As a toner, a polymerized toner obtained by suspension polymerization, emulsion polymerization aggregation method, and the like may be used in addition to a pulverized toner. In particular, in a case of the polymerized toner, a toner having a small particle diameter which has an average diameter of about 4 to 8 μm may be used. The shape of the toner is approximate to a spherical shape. Thus, a toner having a shape which is out from a potato-like spherical shape may be used. The polymerized toner is excellent in charging uniformity and transferability, and is suitably used for increasing image quality.

In a transfer process, an electrostatic transfer method, a pressure transfer method, or an adhesive transfer method, for example, corona transfer, roller transfer, or belt transfer is used. Regarding fixing, thermal roller fixing, flash fixing, oven fixing, pressure fixing, or the like is used.

As cleaning, for example, a brush cleaner, a magnetic brush cleaner, an electrostatic brush cleaner, a magnetic roller cleaner, a blade cleaner, and the like are exemplified.

An erasing process is omitted in many cases. However, in a case of using the erasing process, a fluorescent lamp, an LED, or the like is used. Regarding intensity, exposing energy which is equal to or more than three times that of exposure light is used in many cases. In addition to the above processes, processes of a pre-exposure process and an auxiliary charging process may be provided.

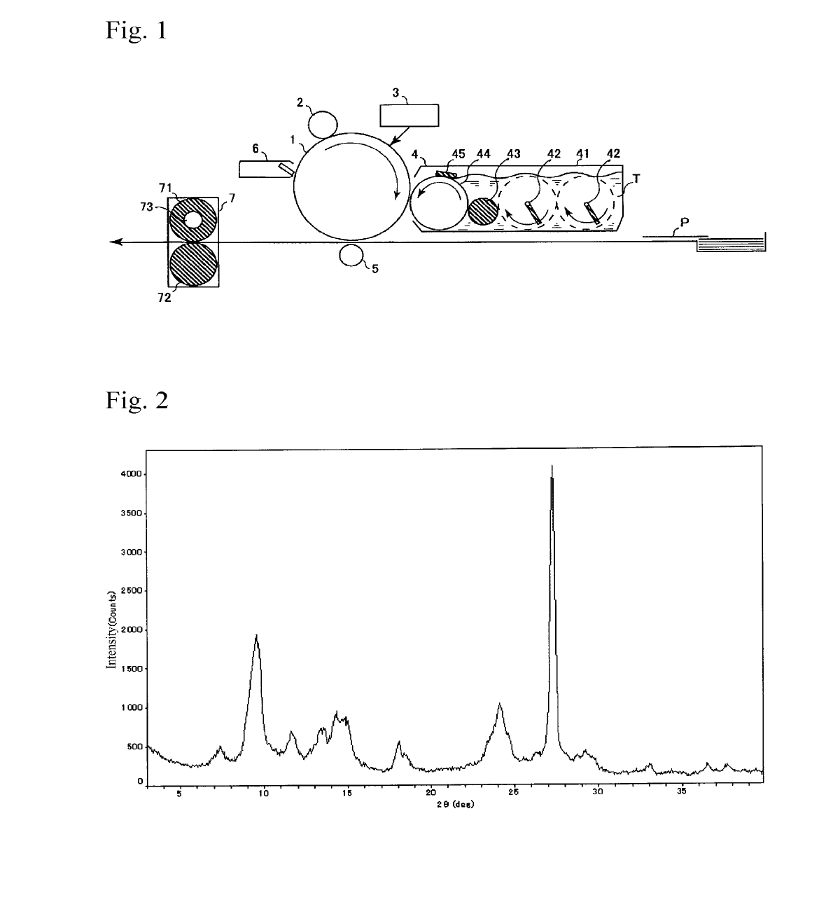

An embodiment of an image forming apparatus which uses the electrophotographic photoreceptor according to the present invention will be described with reference to FIG. 1 which illustrates the main configuration of the apparatus. The embodiment is not limited to the following descriptions, and certain modification thereof in a range without departing from the gist of the present invention may be conducted.

As illustrated in FIG. 1, the image forming apparatus includes an electrophotographic photoreceptor 1, a charging device 2, an exposure device 3, and a developing device 4. If necessary, the image forming apparatus further includes a transfer device 5, a cleaning device 6, and a fixing device 7.

The electrophotographic photoreceptor 1 is not particularly limited as long as the electrophotographic photoreceptor 1 is the above-described electrophotographic photoreceptor according to the present invention. FIG. 1 illustrates a drum-like photoreceptor in which the above-described photosensitive layer is formed on the surface of a cylindrical conductive support, as an example. The charging device 2, the exposure device 3, the developing device 4, the transfer device 5, and the cleaning device 6 are disposed along the outer circumferential surface of the electrophotographic photoreceptor 1, respectively.

The charging device 2 charges the electrophotographic photoreceptor 1. Thus, the charging device 2 uniformly charges the surface of the electrophotographic photoreceptor 1 to a predetermined potential. FIG. 1 illustrates a roller-type charging device (charging roller), as an example of the charging device 2. However, a corona charging device such as a corotron and a scorotron, a contact-type charging device such as a charging brush, and the like are used well.

In many cases, the electrophotographic photoreceptor 1 and the charging device 2 are designed so as to be detachable from the main body of the image forming apparatus, as a cartridge (may be referred to as a photoreceptor cartridge below) which includes both of the electrophotographic photoreceptor 1 and the charging device 2.

For example, in a case where the electrophotographic photoreceptor 1 or the charging device 2 is deteriorated, the photoreceptor cartridge may be detached from the main body of an image forming apparatus, and a new photoreceptor cartridge may be mounted in the main body of the image forming apparatus.

In many cases, a toner which will be described later is designed as a form in which the toner is accumulated in a toner cartridge and the toner cartridge is detachable from the main body of an image forming apparatus. In a case where there is no toner in a toner cartridge which is being used, the toner cartridge may be detached from the main body of the image forming apparatus, and a new toner cartridge may be mounted in the main body of the image forming apparatus. Further, a cartridge which includes all of the electrophotographic photoreceptor 1, the charging device 2, and the toner may be also used.

The type of the exposure device 3 is not particularly limited as long as the exposure device 3 exposes the electrophotographic photoreceptor 1 so as to form an electrostatic latent image on a photosensitive surface of the electrophotographic photoreceptor 1. As specific examples, a halogen lamp, a fluorescent lamp, a laser such as a semiconductor laser and a He—Ne laser, an LED, and the like are exemplified.

Exposure may be performed by the in-photoreceptor exposure type. Light used when exposure is performed is not particular. For example, exposure may be performed by monochromatic light having a wavelength of 780 nm, monochromatic light having a slightly-short wavelength tendency in a region of 600 nm to 700 nm, and monochromatic light having a short wavelength of 380 nm to 500 nm.

The type of a toner T is not particular. A polymerized toner obtained by using a suspension polymerization method, an emulsion polymerization method, and the like may be used in addition to a powder-like toner. In particular, in a case of the polymerized toner, a toner having a small particle diameter, that is, having a diameter of about 4 to 8 μm is preferable. In addition, the shape of particles in the toner is approximate to a spherical shape. Thus, a toner having a shape which is out from a potato-like spherical shape may be also variously used. The polymerized toner is excellent in charging uniformity and transferability, and is suitably used for increasing image quality.

The type of the transfer device 5 is not particularly limited. A device which uses any method such as an electrostatic transfer method a pressure transfer method, or an adhesive transfer method, for example, corona transfer, roller transfer, or belt transfer may be used. Here, the transfer device 5 is set to be configured from a transfer charger, a transfer roller, a transfer belt, and the like which are disposed to oppose the electrophotographic photoreceptor 1. A predetermined voltage value (transfer voltage) which has a polarity reverse to that of the charging potential of the toner T is applied to the transfer device 5, and thus the transfer device 5 transfers a toner image formed on the electrophotographic photoreceptor 1, to recording paper (sheet, medium) P.

The cleaning device 6 is not particularly limited. Any cleaning device such as a brush cleaner, a magnetic brush cleaner, an electrostatic brush cleaner, a magnetic roller cleaner, and a blade cleaner may be used. However, in the present invention, the effect is easily exhibited in a case of the blade cleaner. In the cleaning device 6, a residual toner adhering to the photoreceptor 1 is scraped off by a cleaning member, and the residual toner is restored.

The fixing device 7 is configured from an upper fixing member (fixing roller) 71 and a lower fixing member (fixing roller) 72. A heating device 73 is provided in the fixing member 71 or 72. FIG. 1 illustrates an example in which the heating device 73 is provided in the upper fixing member 71. Each of the upper and lower fixing members 71 and 72 may use well-known thermal fixing members such as a fixing roll in which a metal tube of stainless steel, aluminum, or the like is coated with silicon rubber, a fixing roll in which the metal tube is coated with TEFLON (registered trademark) resin, and a fixing sheet. Further, the fixing members 71 and 72 may have a configuration in which a releasing agent such as silicone oil is supplied in order to improve release properties, or may have a configuration in which a spring and the like causes the fixing members 71 and 72 to forcibly apply pressure to each other.

When a toner transferred onto recording paper P passes through a space between the upper fixing member 71 and the lower fixing member 72 which are heated to a predetermined temperature, the toner is heated until the toner is in a molten state. After passing, the toner is cooled so as to fix the toner onto the recording paper P. The type of the fixing device is not particularly limited. A fixing device by any method, for example, heating roller fixing, flash fixing, oven fixing, or pressure fixing may be provided in addition to the fixing device used here.

In an electrophotographic apparatus configured as described above, recording an image is performed in the following manner. That is, firstly, the surface (photosensitive surface) of the photoreceptor 1 is charged to be a predetermined potential (for example, −600 V), by the charging device 2. At this time, the surface thereof may be charged by a DC voltage or may be charged by a voltage which is obtained by superimposing an AC voltage on a DC voltage.

Then, the charged photosensitive surface of the photoreceptor 1 is exposed by the exposure device 3, in accordance with an image to be recorded. Thus, an electrostatic latent image is formed on the photosensitive surface. The electrostatic latent image formed on the photosensitive surface of the photoreceptor 1 is developed by the developing device 4.

In the developing device 4, a restriction member (developing blade) 45 causes the thickness of a layer formed by the toner supplied by a supply roller 43, to be thin. In addition, the developing device 4 performs friction charging to have a predetermined polarity. While the toner T is held on a developing roller 44, the toner T is transported, and thus is brought into contact with the surface of the photoreceptor 1.

If the charged toner T which has been held on the developing roller 44 is brought into contact with the surface of the photoreceptor 1, a toner image corresponding to the electrostatic latent image is formed on the photosensitive surface of the photoreceptor 1. The toner image is transferred to recording paper P by the transfer device 5. Then, a toner which is not transferred but remains on the photosensitive surface of the photoreceptor 1 is removed by the cleaning device 6.

After the toner image is transferred onto the recording paper P, the toner image is caused to pass through the fixing device 7, and is thermally fixed onto the recording paper P. Thus, a final image is obtained.

The image forming apparatus may have a configuration in which, for example, an erasing process is performed, in addition to the above-described configuration. The erasing process is a process in which exposure is performed to an electrophotographic photoreceptor, and thus erasing is performed on the electrophotographic photoreceptor. As an erasing device, a fluorescent lamp, an LED, or the like is used. Regarding intensity of light used in the erasing process, exposing energy which is equal to or more than three times that of exposure light is used in many cases.

The image forming apparatus may be configured by modification. For example, a configuration in which processes of a pre-exposure process, an auxiliary charging process, and the like can be performed, a configuration in which offset printing is performed, and a configuration of a full-color tandem type using plural types of toners may be made.

The present invention will be described further below in detail by using specific examples. However, the present invention is not limited to the following examples. “A part” in the examples indicates “part by mass”.

### EXAMPLES

<Manufacturing of Dispersion Liquid 1 for Coating and Forming Blocking Layer>

Rutile type white titanium oxide [product name: TTO55N, manufactured by Ishihara Sangyo Corporation] which was treated with 3% of methyldimethoxysilane, and had an average primary particle diameter of 40 nm was dispersed in a methanol solvent for 5 hours, by a ball mill. Thus, a titanium oxide dispersion slurry was obtained.

The titanium oxide dispersion slurry, a solvent mixture of methanol/1-propanol/toluene, and a pellet of a copolymerized polyamide resin were stirred and mixed with being heated, so as to dissolve the polyamide pellet. The copolymerized polyamide resin is formed from ε-caprolactam [compound represented by the following Formula (A)]/bis(4-amino-3-methylcyclohexyl)methane [compound represented by the following Formula (B)]/hexamethylenediamine [compound represented by the following Formula (C)]/decamethylenedicarboxylic acid [compound represented by the following Formula (D)]/octadecamethylene dicarboxylic acid [compound represented by the following Formula (E)], at a composition molar ratio of 60 mol %/15 mol %/5 mol %/15 mol %/5 mol %. Then, ultrasonic dispersion treatment was performed for one hour, and the resultant obtained by the ultrasonic dispersion treatment was filtered by a PTFE membrane filter (Mitex LC manufactured by Advantech Co., Ltd.) having a hole diameter of 5 μm. Thus, a dispersion liquid 1 for coating and forming a blocking layer in which titanium oxide/copolymerized polyamide was provided at a mass ratio of 3/1, a solvent mixture of methanol/1-propanol/toluene at a mass ratio of 7/1/2 was provided, and concentration of solid to be contained was 18.0 mass % was obtained.

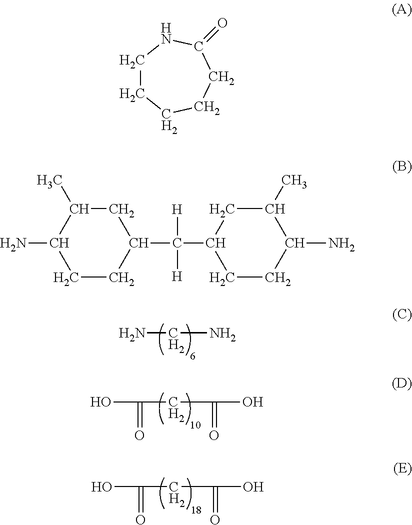

<Manufacturing of Coating Liquid for Forming Charge Transport Layer>

(Preparation Example of Coating Liquid 1-1 for Charge Transport Layer)

Silica particles (manufactured by Nippon Shokubai Co., Ltd., KE-S100 which is a product name was surface-treated) which had been surface-treated with hexamethyldisilazane, and had an average primary particle diameter of 0.8 μm were subjected to ultrasonic dispersion in a tetrahydrofuran solvent for 3 hours. Thus, a silica particle slurry was obtained.

A bisphenol Z type polycarbonate resin (viscosity-average molecular weight Mv=40,000), a charge transport material (CT1) having the following structure, a hydrocarbon compound (HC1) having the following structure, an electron attracting compound (EW1) having the following structure, an oxidant inhibitor (manufactured by BASF Ltd., product name: Irganox1076), and silicone oil (manufactured by Shin-Etsu silicones Co., Ltd., product name: KF-96) were dissolved and prepared in a tetrahydrofuran solvent and an anisole solvent. It was confirmed that the silica particle slurry manufactured in the above process were in the resultant of the dissolving, in a uniform state. Then, mixing was performed.

Accordingly, a coating liquid (1-1) for forming a charge transport layer in which the mass ratio of binder resin/charge transport material/silica/hydrocarbon compound/electron attracting compound/oxidant inhibitor/silicone oil was 100/60/10/5/2/4/0.05, tetrahydrofuran/anisole was 9/1, and solid concentration was 22% was finally manufactured.

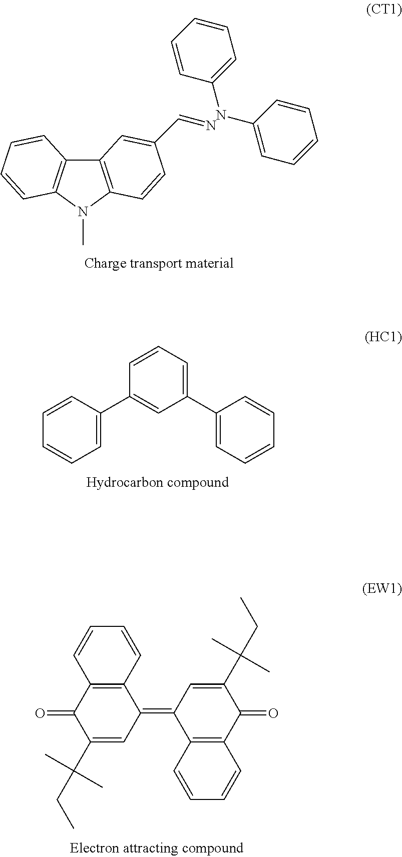

(Coating Liquids 1-2 to 1-6 for Charge Transport Layer)

Coating liquids 1-2 to 1-6 for forming a charge transport layer were manufactured in a similar manner to that of the preparation example of the coating liquid 1-1 for a charge transport layer, but whether or not the silica particles (KE-S100), alumina particles, the hydrocarbon compounds (HC1 and HC2), and the electron attracting compound (EW1) were used was selected. The coating liquids 1-2 to 1-6 were manufactured in details as in the following Table-1.

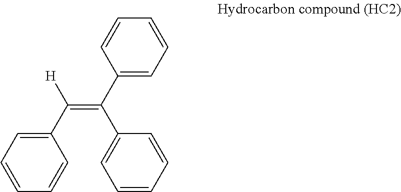 [SMILES: C(=C(c1ccccc1)c1ccccc1)c1ccccc1]

TABLE-1

| | | | Electron | |
|---|---|---|---|---|
| | | | attracting | |
| | Inorganic | Hydrocarbon | compound | Solid |
| | filler | compound | (EW1) | concentration |
| Coating liquid 1-1 | Silica | HC1 | EW1 | 22% |
| Coating liquid 1-2 | Silica | HC1 | None | 22% |
| Coating liquid 1-3 | Silica | None | None | 22% |
| Coating liquid 1-4 | None | HC1 | None | 22% |
| Coating liquid 1-5 | None | None | None | 22% |
| Coating liquid 1-6 | Silica | HC2 | None | 22% |

(Preparation Example of Coating Liquid 2-1 for Charge Transport Layer)

A coating liquid 2-1 for forming a charge transport layer was manufactured by using a coating liquid preparation method which was similar to that for the coating liquid 1-1 for a charge transport layer, except that the following charge transport material CT2 was used instead of the charge transport material CT1 used in the coating liquid 1-1 for a charge transport layer, and following polycarbonate (PC1) (viscosity-average molecular weight Mv=20,000) was used instead of bisphenol Z type polycarbonate. The coating liquid 2-1 had a final coating liquid composition in which the mass ratio of binder resin (PC1)/charge transport material (CT2)/silica (KE-S100)/hydrocarbon compound (HC1)/electron attracting compound (EW1)/oxidant inhibitor (Irganox1076)/silicone oil (KF-96) was 100/55/10/5/2/4/0.05 and solid concentration was 22%.

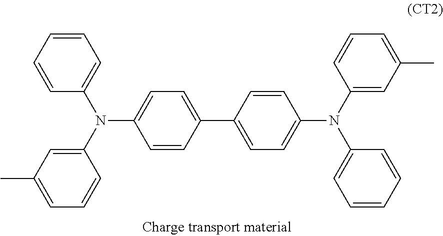

Polycarbonate (PC1)

PC1 is copolymerized polycarbonate of the following structures PA and PB, and PA/PB is 51/49 (molar ratio).

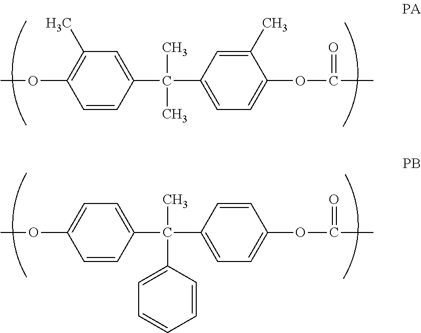

(Coating Liquid 2-2 for Charge Transport Layer)

A coating liquid for forming a charge transport layer was manufactured in a manner that manufacturing was performed in a similar manner to that of the preparation example of the coating liquid 2-1 for a charge transport layer, but the amount of the solvent was adjusted. The manufactured coating liquid had solid concentration of 28%.

<Evaluation of Strong-Exposure Resistance Characteristics of Photoreceptor>

(Manufacturing Method of Test Electrophotographic Photoreceptor)

Regarding the coating liquid in the present invention, in order to evaluate strong-exposure resistance characteristics in a case of being used for a photoreceptor, firstly, an electrophotographic photoreceptor was manufactured by the following method. A dispersion liquid 1 for coating and forming a blocking layer was applied onto a polyethylene terephthalate film which had a surface subjected to aluminum vapor deposition, and had a thickness of 75 μm. Thus, a blocking layer was formed. The blocking layer has a film thickness after dry, which was 1.3 μm.

Then, 10 parts by mass of oxytitanium phthalocyanine were added to 150 parts by mass of 1,2-dimethoxyethane, and grinding dispersion treatment was performed in a sand grinding mill, thereby a pigment dispersion liquid was manufactured. The above oxytitanium phthalocyanine shows a strong diffraction peak at a Bragg angle (2θ±0.2) of 27.3° in X-ray diffraction by a CuKα ray, and has a powder X-ray diffraction spectrum illustrated in FIG. 2.

160 parts by mass of the pigment dispersion liquid obtained in this manner were added to 5 mass % of polyvinyl butyral [manufactured by Denka Ltd., product name: #6000C], and 100 parts by mass of a 1,2-dimethoxyethane solution. 1,2-dimethoxyethane of an appropriate amount was added, and finally a coating liquid for a charge generation layer, in which solid concentration was 4.0 mass % was manufactured. The coating liquid for a charge generation layer was applied onto the above-described blocking layer, by a wire bar, so as to cause a film thickness after dry to be 0.4 μm. Then, drying was performed, thereby a charge generation layer was formed.

Further, the coating liquid for a charge transport layer was applied onto the charge generation layer by using an applicator. After air dry, dry by heating was performed at 125° C. for 20 minutes, so as to cause the film thickness after dry to be the following defined film thickness. Thus, a charge transport layer was provided. In a case of using the coating liquids 1-1 to 1-6 for a charge transport layer, in the manufacturing method of a test electrophotographic photoreceptor, electrophotographic photoreceptors 1-1 to 1-6 were manufactured by setting the film thickness of the charge transport layer to 27 μm.

TABLE-2

| | | | | Coating | Film |
|---|---|---|---|---|---|
| | | | | liquid for | thickness of |
| | | | Electron | charge | charge |
| Photo- | Inorganic | Hydrocarbon | attracting | transport | transport |
| receptor | filler | compound | compound | layer | layer |
| 1-1 | Silica | HC1 | EW1 | 1-1 | 27 μm |
| 1-2 | Silica | HC1 | None | 1-2 | 27 μm |
| 1-3 | Silica | None | None | 1-3 | 27 μm |
| 1-4 | None | HC1 | None | 1-4 | 27 μm |
| 1-5 | None | None | None | 1-5 | 27 μm |
| 1-6 | Silica | HC2 | None | 1-6 | 27 μm |

In a case of using the coating liquids 2-1 and 2-2 for a charge transport layer, in the manufacturing method of a test electrophotographic photoreceptor, electrophotographic photoreceptors 2-1 to 2-3 were manufactured by setting the film thickness of the charge transport layer to be 10 μm, 25 μm, and 45 μm.

TABLE-3

| | | | | Coating | Film |
|---|---|---|---|---|---|
| | | | | liquid for | thickness of |
| | | | Electron | charge | charge |
| Photo- | Inorganic | Hydrocarbon | attracting | transport | transport |
| receptor | filler | compound | compound | layer | layer |
| 2-1 | Silica | HC1 | EW1 | 2-1 | 10 μm |
| 2-2 | Silica | HC1 | EW1 | 2-1 | 25 μm |
| 2-3 | Silica | HC1 | EW1 | 2-2 | 45 μm |

(Evaluation of Strong-Exposure Resistance Characteristics)

The strong-exposure resistance characteristics of each of the obtained photoreceptors were evaluated by evaluating electrical characteristics fluctuation before and after irradiation with strong exposure light. The evaluation method will be described next. Firstly, each of the photoreceptors obtained according to the manufacturing method of a test electrophotographic photoreceptor was mounted in a photoreceptor characteristic test apparatus [manufactured by Mitsubishi Chemical Corporation]. Then, the electrical characteristics were evaluated by a cycle of charging, exposure, potential measurement, and erasing.

That is, charging was performed so as to cause an initial surface potential of the photoreceptor to be −700 V. Exposure was performed by using light which was obtained as monochromatic light of 780 nm from light of a halogen lamp in an interference filter. Then, a surface potential VL at a time of irradiation at 0.4 μJ/cm² was measured. A period from the exposure to the potential measurement was set to 200 milliseconds.

Then, the photoreceptor was irradiated with light of a white fluorescent lamp (Neolumisuper FL20SS·W/18 manufactured by Mitsubishi/Osram Corporation) as strong exposure light. The irradiation was performed for 10 minutes after adjustment was performed so as to cause light intensity on the surface of the photoreceptor to be 2000 lux. After that, the photoreceptor was left at a dark site for 10 minutes, and then the electrical characteristics were evaluated in a manner similar to that performed before the irradiation with the strong exposure light. The surface potential VL was measured.

An absolute value of a changed amount of the surface potential VL before and after irradiation of each electrophotographic photoreceptor with strong exposure light (white fluorescent lamp) was set as ΔVL. As ΔVL becomes smaller, fluctuation of VL was small. Thus, small ΔVL is preferable.

(Evaluation of Photoreceptors 1-1 to 1-7)

Regarding the photoreceptors 1-1 to 1-7, for relative comparison of the absolute value ΔVL of the changed amount of VL, the percentage of ΔVL in each of the photoreceptors when the changed amount ΔVL of VL of the photoreceptor 1-5 is set to 100% is shown in the following Table-4.

TABLE-4

| | ΔVL (relative value when ΔVL of |
|---|---|
| Test photoreceptor | photoreceptor 1-5 is set to 100%) |
| Photoreceptor 1-1 (Example 1) | 41% |
| Photoreceptor 1-2 (Example 2) | 87% |
| Photoreceptor 1-3 (Comparative | 92% |
| Example 1) | |
| Photoreceptor 1-4 (Comparative | 96% |
| Example 2) | |
| Photoreceptor 1-5 (Reference | 100% |
| Example 1) | |
| Photoreceptor 1-6 (Comparative | 96% |
| Example 3) | |

(Evaluation of Photoreceptors 2-1 to 2-3)

Regarding the photoreceptors 2-1 to 2-3, for relative comparison of the absolute value ΔVL of the changed amount of VL, the percentage of ΔVL in each of the photoreceptors when the changed amount ΔVL of VL of the photoreceptor 2-2 is set to 100% is shown in the following Table-5.

TABLE-5

| | | Film thickness |
|---|---|---|
| | | of charge |
| | ΔVL (relative value when | transport |
| | ΔVL of photoreceptor 2-2 | layer in |
| Test photoreceptor | is set to 100%) | photoreceptor |
| Photoreceptor 2-1 | 206% | 10 μm |
| (Comparative Example 4) | | |
| Photoreceptor 2-2 | 100% | 25 μm |
| (Example 3) | | |
| Photoreceptor 2-3 | 188% | 45 μm |
| (Comparative Example 5) | | |

As understood from the above results, it is understood that the photoreceptor in the present invention has good strong-exposure resistance characteristics. Thus, a special labor for handling the photoreceptor is not required, and the photoreceptor can be applied to an image forming apparatus and a drum cartridge.

Evaluation of Mechanical Characteristics of Photoreceptor

(Manufacturing of Coating Liquid and Photoreceptor)

Coating liquids 3-1 and 3-2 for forming a charge transport layer were manufactured in a similar manner to that of the preparation example of the coating liquid 1-1 for a charge transport layer, but whether or not the silica A (obtained by performing surface treatment on KE-S100 by hexamethyldisilazane, particle diameter of 0.8 μm), and the hydrocarbon compound (HC1) were used was selected. The coating liquids 3-1 and 3-2 were manufactured in details as in the following Table-6.

The number of parts by mass of a charge transport agent was set to 50 parts with respect to 100 parts of polycarbonate PC1. The silica particles were changed to silica B (R9200 manufactured by Evonik Corporation, surface-treated with dimethyldichlorosilane, particle diameter of 12 nm), and similarly, coating liquids 4-1 and 4-2 for a charge transport layer were manufactured in details as in the following Table-6. The number of parts by mass of the charge transport agent was set to 50 parts with respect to 100 parts of polycarbonate PC1.

Electrophotographic photoreceptors were manufactured by using the coating liquids for a charge transport layer, in the same manner as that when the electrophotographic photoreceptors 1-1 and 1-2 were manufactured. That is, a blocking layer of 1.3 μm, which used the dispersion liquid 1 for coating and forming a blocking layer, and a charge generation layer were sequentially manufactured on a polyethylene terephthalate film which had a surface subjected to aluminum vapor deposition, and had a thickness of 75 μm. The charge generation layer contains oxytitanium phthalocyanine which shows a strong diffraction peak at a Bragg angle (2θ±0.2) of 27.3° in X-ray diffraction by a CuKα ray, and has a powder X-ray diffraction spectrum illustrated in FIG. 2. Then, electrophotographic photoreceptors shown in Table-7 were manufactured by using the coating liquids 3-1 and 3-2, and 4-1 and 4-2 for a charge transport layer, so as to change the film thickness of the charge transport layer to 25 μm.

TABLE-6

| | | Hydrocarbon | Electron | |
|---|---|---|---|---|
| | Inorganic | compound | attracting | Solid |
| | filler | (HC1) | compound (EW1) | concentration |
| Coating | Silica A | HC1 | None | 23% |
| liquid 3-1 | | | | |
| Coating | Silica A | None | None | 23% |
| liquid 3-2 | | | | |
| Coating | Silica B | HC1 | None | 23% |
| liquid 4-1 | | | | |
| Coating | Silica B | None | None | 23% |
| liquid 4-2 | | | | |

TABLE-7

| Coating | | | | Coating | Film |
|---|---|---|---|---|---|
| liquid for | | | | liquid for | thickness |
| charge | | | Electron | charge | of charge |
| transport | Inorganic | Hydrocarbon | attracting | transport | transport |
| layer | filler | compound | compound | layer | layer |
| 3-1 | Silica A | HC1 | None | 3-1 | 25 μm |
| 3-2 | Silica A | None | None | 3-2 | 25 μm |
| 4-1 | Silica B | HC1 | None | 4-1 | 25 μm |
| 4-2 | Silica B | None | None | 4-2 | 25 μm |

(Evaluation of Mechanical Characteristics)

Each of the manufactured electrophotographic photoreceptors was cut off so as to have a circular shape having a diameter of 10 cm, and abrasion evaluation was performed by a Taber abrasion tester (manufactured by Taber Corporation). Regarding test conditions, the abrasion amount after 1000 times of rotations at a load of 500 g were performed at 23° C. by using a wear wheel CS-10F under an atmosphere of 50% RH was measured by comparing mass before the test to mass after the test. Table-8 shows measurement results.

TABLE-8

| Photoreceptor | Abrasion amount (mg) |
|---|---|
| 3-1 | 3.49 |
| 3-2 | 3.63 |
| 4-1 | 4.29 |
| 4-2 | 4.14 |

As shown in Table-8, the followings are understood. That is, in a case of using silica having a large particle diameter, the abrasion resistance is more excellent than that in a case of using silica having a small particle diameter. However, it is possible to achieve further improvement of abrasion resistance by using the hydrocarbon compound in this application, together.

The present invention is described in detail by using the specific forms. However, it is apparent from the skilled person in the related art that various changes and modifications may be made without departing from the intention and the scope of the present invention. This application is based upon and claims the benefit of priority from Japanese Patent Application No. 2014-241987; filed Nov. 28, 2014; the entire contents of which are incorporated herein by reference.

1. An electrophotographic photoreceptor comprising, on a conductive base:
a charge generation layer; and
a charge transport layer having a film thickness of 15 μm to 40 μm,
wherein
the charge transport layer is an outermost layer, and
the charge transport layer contains an inorganic filler and a hydrocarbon compound represented by the following Formula (1):

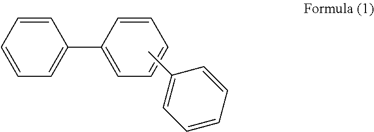

2. The electrophotographic photoreceptor according to claim 1, wherein the inorganic filler is silica.

3. The electrophotographic photoreceptor according to claim 2, wherein the silica is subjected to a surface modification.

4. The electrophotographic photoreceptor according to claim 1, wherein the inorganic filler has an average primary particle diameter of 0.01 μm to 1 μm.

5. The electrophotographic photoreceptor according to claim 1, wherein the charge transport layer contains a binder resin, and a content of the inorganic filler is 5 mass % to 30 mass % with respect to the binder resin.

6. The electrophotographic photoreceptor according to claim 1, wherein a percentage of the hydrocarbon compound represented by Formula (1) is 10 mass % to 100 mass %, with respect to the inorganic filler.

7. The electrophotographic photoreceptor according to claim 1, wherein the charge transport layer contains an electron attracting compound represented by Formula (2): 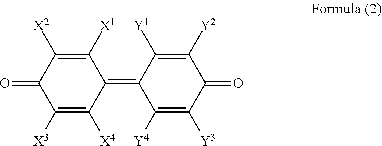
[in Formula (2), X¹, X², X³, X⁴, Y¹, Y², Y³, and Y⁴ each respectively indicate a hydrogen atom, an alkyl group, an aryl group, an acyl group, or a bivalent organic group, and a ring structure including X¹ and X², a ring structure including X³ and X⁴, a ring structure including Y¹ and Y², and a ring structure including Y³ and Y⁴ may be formed]

8. The electrophotographic photoreceptor according to claim 7, wherein the electron attracting compound represented by Formula (2) is any one of compounds represented by the following Formulas (2a) to (2d):

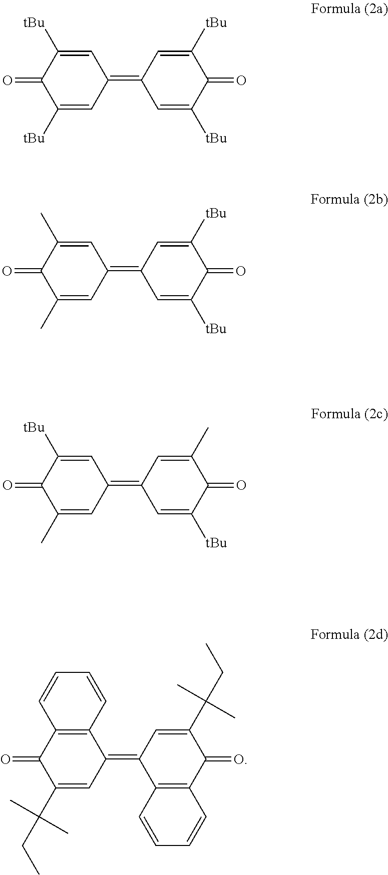 [SMILES: CC(C)(C)C1=CC(=C2C=C(C(C)(C)C)C(=O)C(C(C)(C)C)=C2)C=C(C(C)(C)C)C1=O ; CC1=CC(=C2C=C(C(C)(C)C)C(=O)C(C(C)(C)C)=C2)C=C(C)C1=O ; CC1=C/C(=C2/C=C(C)C(=O)C(C(C)(C)C)=C2)C=C(C(C)(C)C)C1=O ; CCC(C)(C)C1=C/C(=C2/C=C(C(C)(C)CC)C(=O)c3ccccc32)c2ccccc2C1=O]

9. The electrophotographic photoreceptor according to claim 7, wherein a content percentage of the compound represented by Formula (2) is 2 mass % to 50 mass % with respect to the silica.

10. The electrophotographic photoreceptor according to claim 1, wherein the charge generation layer contains D type (Y type) titanyl phthalocyanine in which a clear peak is shown at a Bragg angle 2θ (±0.2°) which is 27.1° to 27.3°, in a CuKα characteristic X-ray diffraction spectrum.

11. The electrophotographic photoreceptor according to claim 1, wherein the charge generation layer contains D type (Y type) titanyl phthalocyanine in which the maximum peak is provided at at least a Bragg angle 2θ±0.2° which is 27.2° and a peak is not provided at 26.2° in a CuKα characteristic X-ray diffraction spectrum, and a peak regarding a temperature change from 50° C. to 400° C., other than a peak by vaporization of absorption water is not provided in differential scanning calorimetry.

12. The electrophotographic photoreceptor according to claim 1, further comprising a blocking layer.
